(19) United States
(12) Patent Application Publication
(10) Pub. No.: US 2026/0174952 A1
(43) Pub. Date: Jun. 25, 2026

(54) Wearable Drug Delivery Device

(71) Applicant: TxSphere, LLC, Natick, MA (US)

(72) Inventor: Zhenhua Mao, Andover, MA (US)

(21) Appl. No.: 19/425,455

(22) Filed: Dec. 18, 2025

Related U.S. Application Data
(60) Provisional application No. 63/736,993, filed on Dec. 20, 2024.

(51) Int. Cl. A61M 5/142

(52) U.S. Cl. CPC A61M 5/14228; A61M 5/14248; A61M 2005/14252; A61M 2205/3389

(57) ABSTRACT

A needle injection mechanism for a wearable pump assembly includes a drive motor, a drive shaft, and a drive train. A linkage transfers rotation of the drive shaft to linear motion. A first slide block is connected to the linkage and engages a second slide block to move along a guide member responsive to the linear motion. A first end of a stylet is mounted to the fist slide block. A cannula has a first end mounted to the second slide block and a lumen through which a second end of the stylet is slidably inserted. A resilient retention tab is deflected as the second slide block moves across and holds the second slide block in the second position. The first slide block returns to the first position after driving the second slide block to the second position, retracting the stylet from the cannula.

### CROSS-REFERENCE TO RELATED APPLICATIONS

This application claims priority to U.S. Provisional Application Ser. No. 63/736,993, filed Dec. 20, 2024, the entire contents of which is incorporated herein by reference.

### BACKGROUND OF THE INVENTION

### Field of the Invention

The subject matter disclosed herein relates to a wearable device for delivery of therapeutic liquids to a patient. More specifically, a wearable device provides a reusable pump mechanism mountable to a disposable assembly which receives a container containing the therapeutic liquid and which includes a needle injection mechanism for delivering the therapeutic liquid to the patient.

### Description of Related Art

As is known to those skilled in the art, fluid delivery devices have the ability to provide a liquid medicine or other therapeutic liquid, herein referred to as medicaments, to a patient over a period of time. Initially, wearable pumps utilizing straps, harnesses, carrying pouches, and the like provided for infusion pumps that could be carried by a patient. Recent developments have provided for pumps having a reduced size that may be adhered directly to a patient. Such pumps are often single-use devices, including a pre-loaded amount of medicament. The device is secured to the patient, a needle inserted into the patient, and a pump delivers the medicament via the needle to the patient.

However, such wearable pumps have not been fully met without incurring various certain disadvantages. Disposing of each device generates not only waste in the form of the housing for the medicament, but the entire delivery mechanism as well. Each device may include some electronic components, for example, a motor to drive the pump, a display to provide information to the patient, and a controller to control operation of the pump or to provide information on the display.

Thus, it would be desirable to provide a wearable pump which reduces the amount of waste generated per dose.

Wearable pumps also have a challenge for injecting a needle into the patient. In order to reduce electronic components on a wearable device, manual insertion of the needle may be required. While needle insertion devices may be provided which require just the press of a button to insert the needle, where retraction of the button similarly retracts the needle and leaves the cannula inserted, a patient may be reluctant to manually press the button. The patient may also stop pressing the button upon feeling initial insertion of the needle and may not fully inject the needle and/or cannula.

Thus, it would be desirable to provide an improved needle injection mechanism for automatic insertion of a needle into the patient.

### BRIEF DESCRIPTION OF THE INVENTION

The subject matter disclosed herein describes a wearable pump which reduces the amount of waste generated per dose. The wearable pump includes a first housing and a second housing, where the second housing is removably mounted to the first housing. The first housing includes components which, for example, require sterilization and/or which may be in contact with a patient or with medicament during delivery of a dose, such as a cartridge, a vial, or a bag of the medicament; a cannula assembly for subcutaneous delivery of the medicament; and tubing connecting the cartridge to the cannula assembly. The second housing includes components used to control operation of the wearable pump, such as a control board, a motor, a pump, a battery, and the like. The components in the first housing are single-use components, and the components in the second housing are reused in combination with multiple first housings to deliver multiple doses.

The subject matter disclosed herein further describes a wearable pump which provides an improved needle injection mechanism for automatic insertion of a needle into the patient. The needle injection mechanism is split between the first housing and the second housing. A needle slide mechanism is provided in the first housing which permits the cannula assembly to move between a retracted and an extended position. A drive mechanism is provide in the second housing to activate the needle slide mechanism and to automatically insert a cannula into a patient for delivery of the medicament.

According to one embodiment of the invention, a wearable pump assembly includes a housing to be carried on a body of a patient. A reservoir is in fluid communication with the housing and is configured to hold a fluid for delivery from the pump assembly. A pump is mounted within the housing and configured to transfer the fluid from the reservoir to a cannula. The wearable pump assembly also includes a needle injection mechanism with a drive motor operative to rotate a drive shaft and a drive train operative to transfer rotation of the drive shaft to a linear motion via a linkage. The needle injection mechanism also includes a first slide block, a second slide block, and a retention tab made of a resilient material. The first slide block is connected to the linkage to move along a guide member between a first position and a second position responsive to the linear motion, and the first slide block slidably moves a stylet through a lumen of the cannula while moving between the first position and the second position. The second slide block is pushed by the first slide block between a first position and a second position along the guide member. The retention tab is deflected from an initial position to a deflected position as the second slide block moves from the first position to the second position of the second slide block, and the retention tab returns to the initial position to hold the second slide block in the second position when the second slide block reaches the second position. The first slide block returns to the first position of the first slide block after pushing the second slide block to the second position of the second slide block, retracting the stylet from the lumen of the cannula.

According to aspects of the invention, at least one tube extends between the reservoir and the second slide block. The second slide block includes a first opening in which the at least one tube is inserted, a second opening in which the cannula is inserted, and a passage extending between the first opening and the second opening to deliver the fluid from the at least one tube to the cannula. The stylet is inserted through the passage and into the lumen of the cannula as the first slide block moves between the first position and the second position of the first slide block, and a second end of the stylet is retracted from the passage when the first slide block returns to the first position of the first slide block and the second slide block is retained in the second position of the second slide block.

According to still other aspects of the invention, the second opening is on a front surface of the second slide block, and the second slide block includes a third opening on a rear surface of the second slide block opposite the second opening. The needle injection mechanism further comprises an insert block sized to fit the third opening, where the insert block includes a passage for the stylet, the stylet slidably engages the passage in the insert block, the insert block seals the third opening, and the insert block provides a seal around the stylet as the stylet slides within the passage.

According to yet another aspect of the invention, the pump is a peristaltic pump including a plurality of fingers operative to engage the at least one tube to draw the fluid from the reservoir to the second slide block. The drive train of the needle injection mechanism may include a drive gear mounted to the drive shaft, multiple additional gears sequentially driven from the drive gear, and a flywheel coaxially mounted with one of the additional gears to rotate when the drive motor rotates the drive shaft. The linkage is a drive arm having a first end pivotally mounted to the flywheel and a second end pivotally mounted to the first slide block.

According to another embodiment of the invention, a needle injection mechanism for a wearable pump assembly includes a drive motor operative to rotate a drive shaft and a drive train operative to transfer rotation of the drive shaft to motion of a linkage, where the linkage is operative to convert rotational motion from the drive train to a linear motion. A first slide block is operative to move between a first position and a second position along a guide member responsive to the linear motion, and a stylet having a first end is mounted to the fist slide block. A second slide block is operative to move between a first position and a second position along the guide member. The first slide block engages the second slide block as the first slide block moves between the first position and the second position of the first slide block to drive the second slide block from the first position to the second position of the second slide block. A cannula has a first end mounted to the second slide block and a lumen through which a second end of the stylet is slidably inserted. The needle injection mechanism also includes a retention tab made of a resilient material, where the retention tab is deflected from an initial position to a deflected position as the second slide block moves from the first position to the second position and the retention tab returns to the initial to hold the second slide block in the second position when the second slide block reaches the second position. The first slide block returns to the first position after driving the second slide block to the second position, retracting the stylet from the cannula.

These and other advantages and features of the invention will become apparent to those skilled in the art from the detailed description and the accompanying drawings. It should be understood, however, that the detailed description and accompanying drawings, while indicating preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

Various exemplary embodiments of the subject matter disclosed herein are illustrated in the accompanying drawings in which like reference numerals represent like parts throughout, and in which:

FIG. 1 is a perspective view of a wearable cartridge pump according to one embodiment of the invention;

FIG. 2 is a front plan view of the wearable cartridge pump of FIG. 1;

FIG. 3 is a side elevational view of the wearable cartridge pump of FIG. 1;

FIG. 4 is a rear plan view of the wearable cartridge pump of FIG. 1;

FIG. 5 is a perspective view of the wearable cartridge pump of FIG. 1 with the housing removed;

FIG. 6 is a front plan view of the wearable cartridge pump of FIG. 5;

FIG. 7 is a right side elevational view of the wearable cartridge pump of FIG. 5;

FIG. 8 is a rear view of the wearable cartridge pump of FIG. 5;

FIG. 9 is a left side elevational view of the wearable cartridge pump of FIG. 5;

FIG. 10 is a perspective view of a first housing for the wearable cartridge pump of FIG. 1;

FIG. 11 is a bottom elevation view of the first housing of FIG. 10;

FIG. 12 is a perspective view of a second housing for the wearable cartridge pump of FIG. 1 from the front, bottom, and first side;

FIG. 13 is a bottom elevation view of the second housing of FIG. 12

FIG. 14 is a perspective view of a second housing for the wearable cartridge pump of FIG. 1 from the front, first side, and top;

FIG. 15 is a top elevation view of the second housing of FIG. 14;

FIG. 16 is a perspective view of a needle injection mechanism for the wearable cartridge pump of FIG. 5;

FIG. 17 is a bottom plan view of the needle injection mechanism of FIG. 16;

FIG. 18 is a partial perspective view of the needle injection mechanism of FIG. 16;

FIG. 19 is another partial perspective view of the needle injection mechanism of FIG. 16 with the needle and cannula fully retracted;

FIG. 20 is a sectional view of the needle injection mechanism of FIG. 16 with the needle and cannula fully retracted;

FIG. 21 is another partial perspective view of the needle injection mechanism of FIG. 16 with the needle and cannula fully extended;

FIG. 22 is a sectional view of the needle injection mechanism of FIG. 16 with the needle and cannula fully extended;

FIG. 23 is another partial perspective view of the needle injection mechanism of FIG. 16 with the needle retracted after insertion and the cannula extended in the insertion position;

FIG. 24 is a sectional view of the needle injection mechanism of FIG. 16 with the needle retracted after insertion and the cannula extended in the insertion position;

FIG. 25 is a rear view of a flywheel, transfer plate, and guide plate in a retracted position for the needle injection mechanism of FIG. 16;

FIG. 26 is a rear view of a flywheel, transfer plate, and guide plate in an extended position for the needle injection mechanism of FIG. 16;

FIG. 27 is a partial perspective view of the needle injection mechanism illustrating a needle and a cannula;

FIG. 28 is a perspective view of a cannula for the needle injection mechanism of FIG. 16 according to one embodiment of the invention;

FIG. 29 is a sectional view of the cannula of FIG. 28;

FIG. 30 is a partial sectional view of the cannula assembly, needle slide, and cannula slide of the needle injection mechanism of FIG. 16 with both the needle and cannula retracted;

FIG. 31 is a partial sectional view of the cannula assembly, needle slide, and cannula slide of the needle injection mechanism of FIG. 16 with both the needle and cannula extended;

FIG. 32 is a partial sectional view of the cannula assembly, needle slide, and cannula slide of the needle injection mechanism of FIG. 16 with the needle retracted and the cannula extended;

FIG. 33 is a perspective view of the second housing prior to engaging a trigger mechanism for a cartridge assembly in the wearable cartridge pump of FIG. 1;

FIG. 34 is a front plan view of the second housing prior to engaging the trigger mechanism for the cartridge assembly of FIG. 33;

FIG. 35 is a bottom elevational view of the second housing prior to engaging the trigger mechanism for the cartridge assembly of FIG. 33;

FIG. 36 is a side elevational view of the second housing prior to engaging the trigger mechanism for the cartridge assembly of FIG. 33;

FIG. 37 is a partial section view of the cartridge assembly in the wearable cartridge pump of FIG. 33 prior to the second housing engaging the trigger mechanism;

FIG. 38 is a perspective view of the second housing after engaging the trigger mechanism for the cartridge assembly in the wearable cartridge pump of FIG. 33;

FIG. 39 is a front plan view of the second housing after engaging the trigger mechanism for the cartridge assembly of FIG. 38;

FIG. 40 is a bottom elevational view of the second housing after engaging the trigger mechanism for the cartridge assembly of FIG. 38;

FIG. 41 is a side elevational view of the second housing after engaging the trigger mechanism for the cartridge assembly of FIG. 38;

FIG. 42 is a partial section view of the cartridge assembly in the wearable cartridge pump of FIG. 38 after the second housing engaging the trigger mechanism;

FIG. 43 is an exemplary environmental view illustrating the wearable cartridge pump mounted on a patient;

FIG. 44 is a partial exploded view of the wearable cartridge pump of FIG. 43;

FIG. 45 is a partial bottom elevation view of one embodiment of a fluid flow path within the wearable cartridge pump of FIG. 1;

FIG. 46 is a partial top plan view of the fluid flow path of FIG. 45;

FIG. 47 is a perspective view of a wearable cartridge pump according to another embodiment of the invention;

FIG. 48 is a perspective view of the wearable cartridge pump of FIG. 47 with a portion of the housing removed;

FIG. 49 is a bottom elevation view of the wearable cartridge pump of FIG. 48;

FIG. 50 is a front plan view of the wearable cartridge pump of FIG. 48;

FIG. 51 is a partial perspective view of a needle injection mechanism from the wearable cartridge pump of FIG. 47 with the needle injection mechanism in an initial position;

FIG. 52 is a partial perspective view of the needle injection mechanism from the wearable cartridge pump of FIG. 47 with the needle injection mechanism in an extended position to insert the needle and cannula into a patient;

FIG. 53 is a partial perspective view of a needle injection mechanism from the wearable cartridge pump of FIG. 47 with the cannula in an insertion position and the needle in a retracted position;

FIG. 54 is a partial cutaway view of the needle injection mechanism from the wearable cartridge pump of FIG. 47 with the cannula in an insertion position and the needle in a retracted position;

FIG. 55 is a sectional view of a second slide block for the needle injection mechanism of FIG. 47;

FIG. 56 is a partial exploded view of the second slide block of FIG. 55;

FIG. 57 is a perspective view of the second slide block of FIG. 55;

FIG. 58 is a side elevational view of the second slide block of FIG. 55; and

FIG. 59 is a front plan view of the second slide block of FIG. 55.

In describing the various embodiments of the invention which are illustrated in the drawings, specific terminology will be resorted to for the sake of clarity. However, it is not intended that the invention be limited to the specific terms so selected and it is understood that each specific term includes all technical equivalents which operate in a similar manner to accomplish a similar purpose. For example, the word “connected,” “attached,” or terms similar thereto are often used. They are not limited to direct connection but include connection through other elements where such connection is recognized as being equivalent by those skilled in the art.

### DETAILED DESCRIPTION OF THE INVENTION

The various features and advantageous details of the subject matter disclosed herein are explained more fully with reference to the non-limiting embodiments described in detail in the following description.

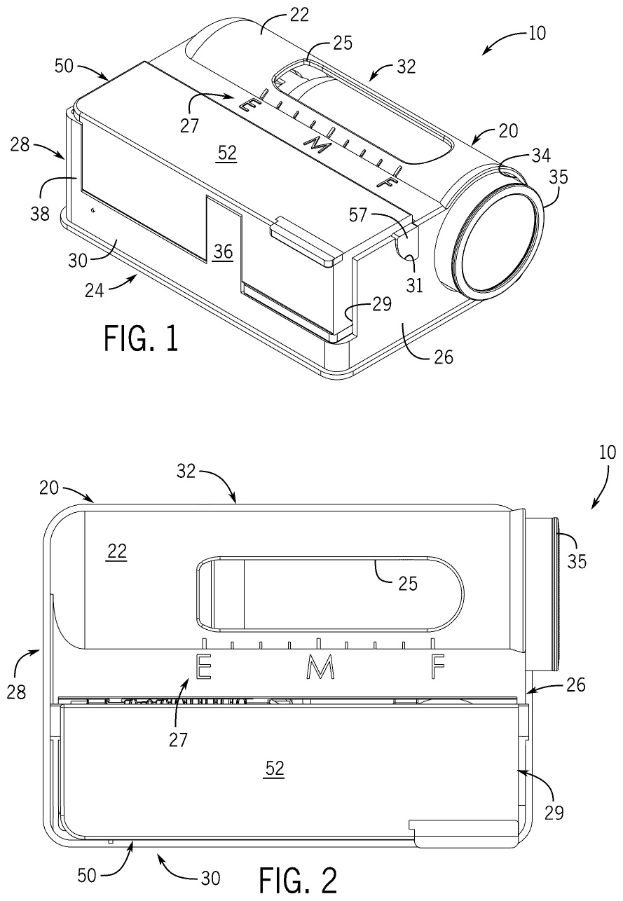

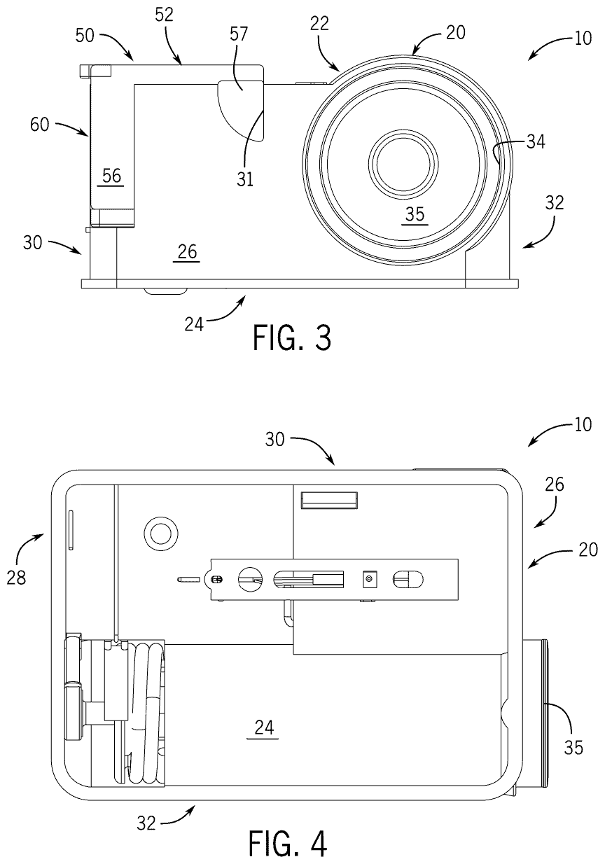

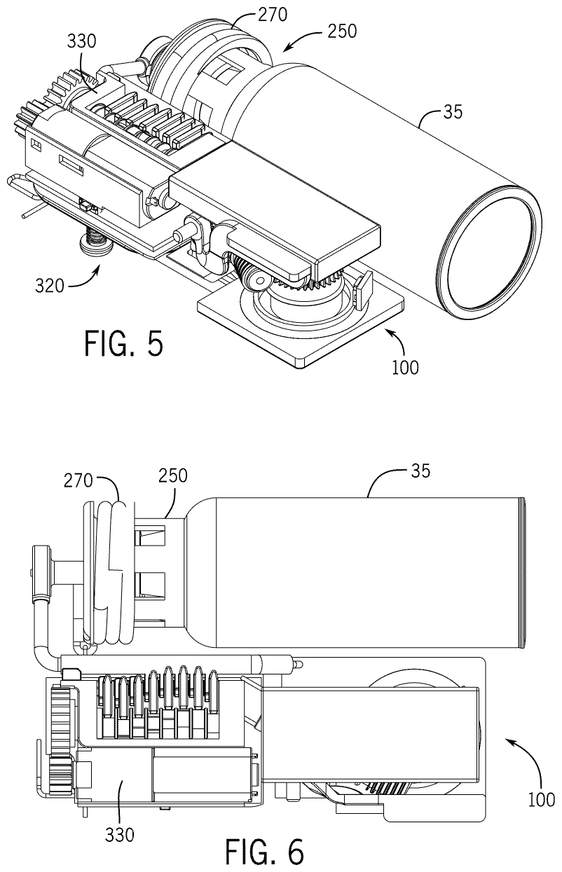

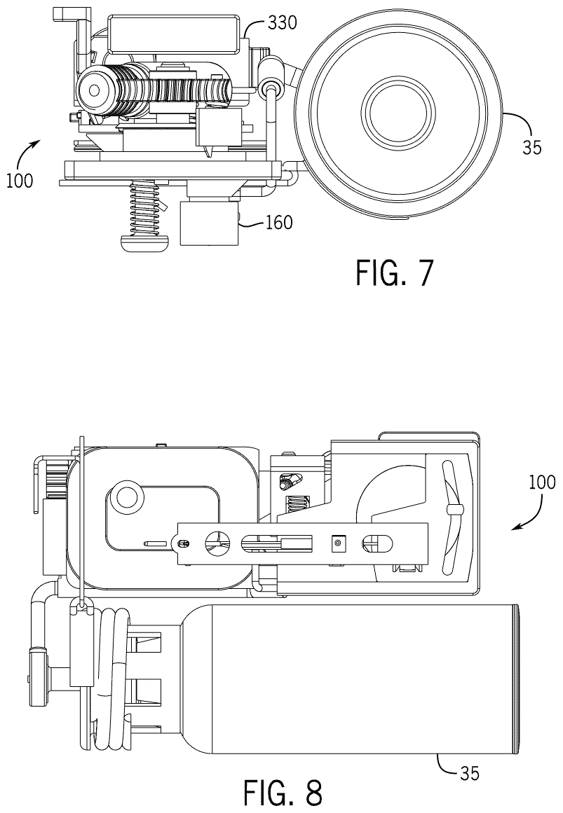

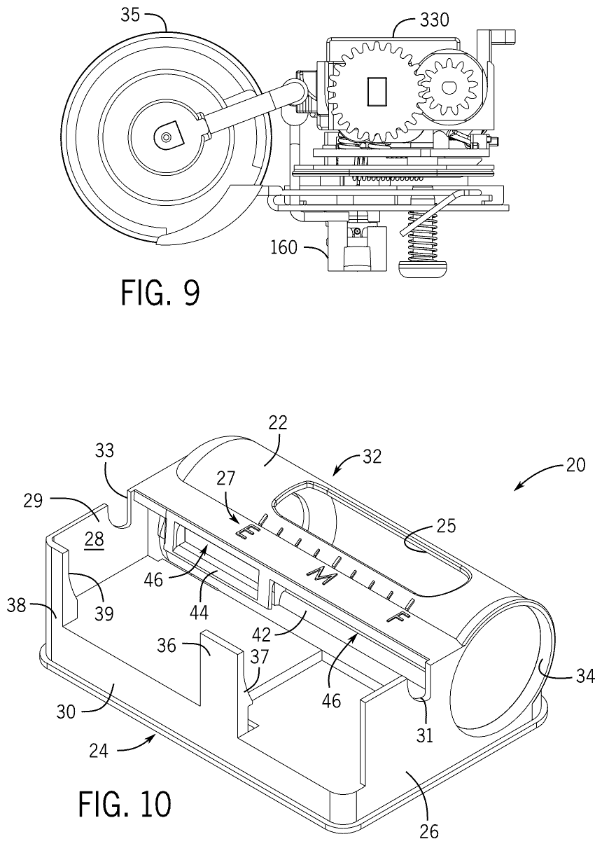

Turning initially to FIGS. 1-4, a wearable cartridge pump 10 according to one embodiment of the invention is illustrated. The wearable cartridge pump 10 includes two portions. A first portion is configured to receive medicament for delivery to a patient, and a second portion is configured to hold a pump 330 for delivering the medicament to the patient. The first portion is contained within a first housing 20 and is configured to be disposable, or single-use. The second portion is contained within a second housing 50 and is configured to be reusable, or multi-use. The second housing 50 is configured to be inserted into a volume 29 within the first housing 20 to deliver the medicament and to be removed from the first housing when delivery of the medicament is complete. When the second housing 50 is mounted within the first housing 20, the wearable cartridge pump 10 is, generally, box-like in form.

The cartridge pump 10 includes a reservoir to hold the medicament for delivery to the patient. According to one aspect of the invention, the reservoir is integrated within the first housing 20. The first housing 20 may include a fill port for insertion of the medicament into the reservoir. According to another aspect of the invention, the reservoir is a cartridge 35 which is insertable into the first housing 20. The cartridge 35 may be filled and sealed by a drug supplier for subsequent distribution. Optionally, the cartridge 35 may be filled by a pharmacist, pharmacy technician, or other such personnel from a larger supply of medicament. Whether pre-filled by the manufacturer or filled at a distribution point, the cartridge 35 is then insertable into the first housing 20. According to yet another aspect of the invention, because the first housing 20 is intended to be a single-use item and disposable, a cartridge may be inserted into the first housing 20 by a drug manufacturer, and the first housing 20 in combination with the cartridge 35 is distributed to patients.

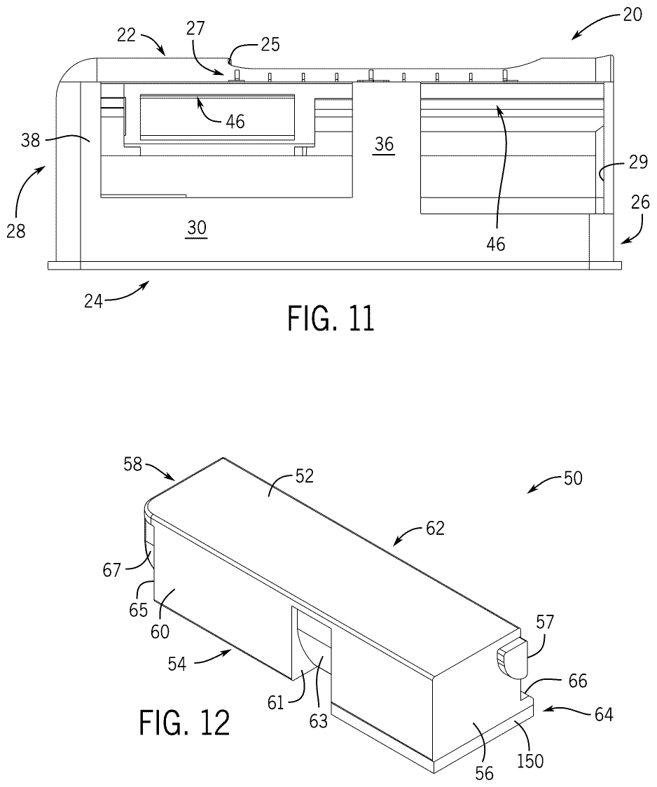

The first housing 20 includes a front side 22 and a rear side 24, opposite the front side; a first side 26 and a second side 28, opposite the first side; and a bottom side 30 and a top side 32, opposite the bottom side. The front side 22 includes a first opening 25 through which the cartridge 35 may be observed. The front side 22 further includes indicia 27 along the first opening 25 providing an indication to the patient of the amount of medicament remaining in the cartridge 35. The bottom side 30 has a generally planar surface extending from the rear side 24 toward the front side 22 for a portion of a height of the first housing 20. The generally planar surface ends where the volume 29 for receiving the second housing 50 begins. The bottom side 30 also includes a pair of tabs 36, 38 extending from the edge of the planar surface for a remainder of the distance to the front side 22. The first tab 36 is generally positioned in the middle of the bottom side and has a width less than a height. The second tab 38 is located along an edge between the bottom side 30 and the second side 28 of the first housing 20. The tabs 36, 38 are configured to help retain the second housing 50 within the volume 29 of the first housing 20. With reference also to FIGS. 10 and 11, an interior surface of the first tab 36 includes a first curved surface 37, and an interior surface of the second tab 38 includes a second curved surface 39. The first and second curved surfaces 37, 39 are complementary to curved surfaces on the second housing 50 and facilitate insertion of the second housing into the volume 29 of the first housing 20. The first side 26 includes a first curved slot 31, and the second side 28 includes a second curved slot 33. Each of the first and second curved slots 31, 33 are configured to receive complementary curved tabs 57, 59 on the second housing 50 to further facilitate insertion of the second housing into the volume 29 of the first housing 20.

Relational terms used herein are not intended to be limiting. Rather, the relational terms are used to describe elements within a figure and describe relationships between elements as illustrated in the figure. Terms such as upper and lower, left and right, front and back, side, top, bottom, and the like may change by rotation of the wearable cartridge pump 10.

According to one aspect of the invention, the cartridge 35 is clear or translucent such that a level of medicament remaining in the cartridge is visible through the cartridge. Initially, the level of medicament is proximate the F indicia 27, indicating that the cartridge is full. As medicament is drawn from the cartridge 35, the level moves from the F indicia 27 to the M and then to the E indicia 27, indicating a middle level and an empty level, respectively. Alternate indicia, such as one hundred percent, fifty percent, and zero percent or one, one-half, and zero, and the like may be utilized to indicate a fill level. According to another aspect of the invention, a plunger mechanism may be utilized to empty the cartridge. The position of the plunger may be viewed through the cartridge 35 and be utilized in combination with the indicia 27 to indicate a level of medicament remaining in the cartridge. Optionally, the cartridge 35 may be opaque such that the level of medicament is not visible through the cartridge, an indicator (not shown) may be connected to the plunger and positioned to move along the outside of the cartridge past the indicia 27 to indicate a level of medicament remaining in the cartridge.

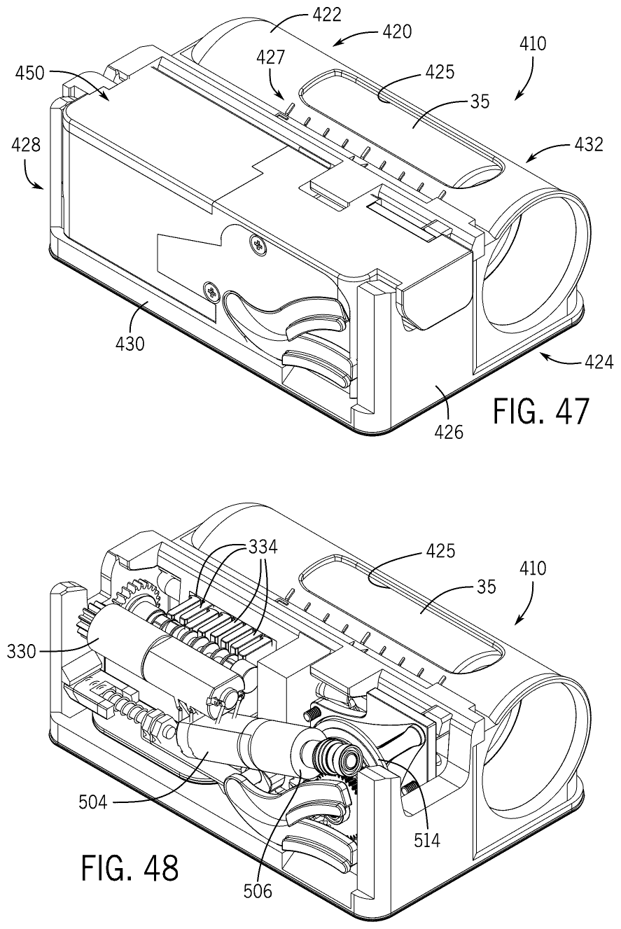

With reference next to FIG. 47, a wearable cartridge pump 410 according to another embodiment of the invention is illustrated. The wearable cartridge pump 410 includes two portions. A first portion is configured to receive medicament for delivery to a patient, and a second portion is configured to hold the pump 330 for delivering the medicament to the patient. The first portion is contained within a first housing 420 and is configured to be disposable, or single-use. The second portion is contained within a second housing 450 and is configured to be reusable, or multi-use. The second housing 450 is configured to be inserted into a volume within the first housing 420 to deliver the medicament and to be removed from the first housing when delivery of the medicament is complete. When the second housing 450 is mounted within the first housing 420, the wearable cartridge pump 410 is, generally, box-like in form.

The cartridge pump 410 includes a reservoir to hold the medicament for delivery to the patient. According to one aspect of the invention, the reservoir is integrated within the first housing 420. The first housing 420 may include a fill port for insertion of the medicament into the reservoir. According to another aspect of the invention, the reservoir is a cartridge 35 which is insertable into the first housing 420. The cartridge 35 may be filled and sealed by a drug supplier for subsequent distribution. Optionally, the cartridge 35 may be filled by a pharmacist, pharmacy technician, or other such personnel from a larger supply of medicament. Whether pre-filled by the manufacturer or filled at a distribution point, the cartridge 35 is then insertable into the first housing 420. According to yet another aspect of the invention, because the first housing 420 is intended to be a single-use item and disposable, a cartridge may be inserted into the first housing 420 by a drug manufacturer, and the first housing 420 in combination with the cartridge 35 is distributed to patients.

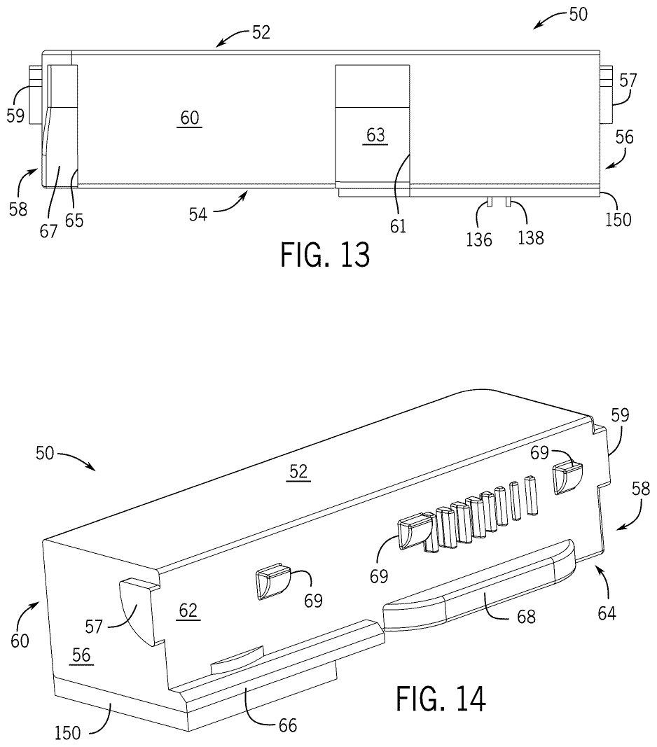

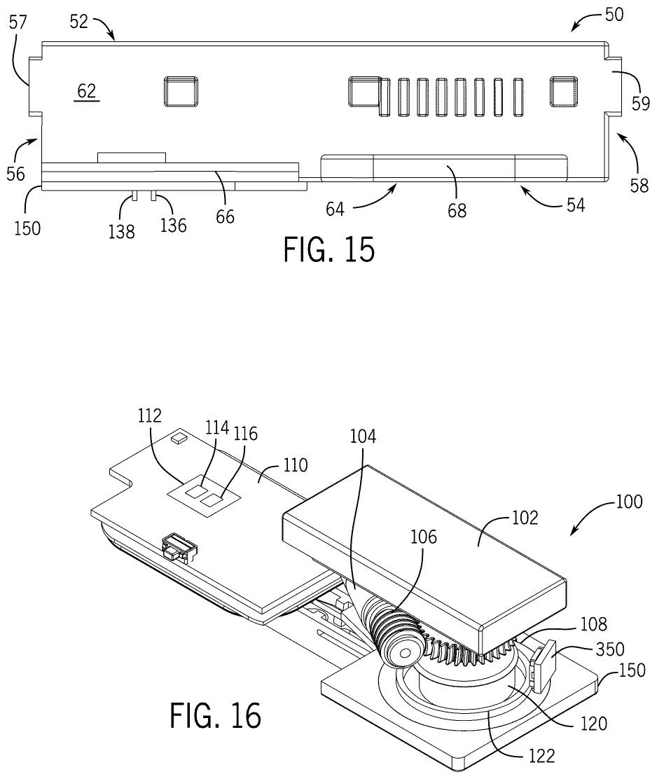

The first housing 420 includes a front side 422 and a rear side 424, opposite the front side; a first side 426 and a second side 428, opposite the first side; and a bottom side 430 and a top side 432, opposite the bottom side. The front side 422 includes a first opening 425 through which the cartridge 35 may be observed. The front side 422 further includes indicia 427 along the first opening 425 providing an indication to the patient of the amount of medicament remaining in the cartridge 35. The bottom side 430 has a generally planar surface extending from the rear side 424 toward the front side 422 for a portion of a height of the first housing 420. The generally planar surface ends where the volume for receiving the second housing 450 begins. With reference also to FIGS. 12-15, one embodiment of a second housing 50 holding a pump 330 for the wearable cartridge pump 10 is illustrated. The second housing 50 is generally in the form of an elongated box. The second housing 50 includes a front side 52 and a rear side 54, opposite the front side; a first side 56 and a second side 58, opposite the first side; and a bottom side 60 and a top side 62, opposite the bottom side. The front side 52 is generally planar, extending between the first and second sides 56, 58 and between the bottom and top sides 60, 62. The first side 56 includes a first tab 57 protruding from the first side and proximate the top side 62 of the second housing 50. The top of the first tab 57 is flat and extends along an edge of the second housing between the first side 56 and the top side 62. The bottom of the first tab 57 is curved and configured to engage the first curved slot 31 on the first side 26 of the first housing 20 as the second housing is inserted into the first housing. The second side 58 includes a second tab 59 protruding from the second side and proximate the top side 62 of the second housing 50. The top of the second tab 59 is flat and extends along an edge of the second housing between the second side 58 and the top side 62. The bottom of the second tab 59 is curved and configured to engage the second curved slot 33 on the second side 28 of the first housing 20 as the second housing is inserted into the first housing. The bottom side 60 of the second housing 50 includes a first recess 61 with a curved surface 63 on the interior of the recess 61. The first recess 61 is positioned along the bottom side 60 such that it aligns with the first tab 36 on the bottom side 30 of the first housing when the second housing 50 is inserted into the volume 29 of the first housing 20. The width of the first recess 61 is configured to receive the first tab 36 on the bottom side 30 of the first housing. The curved surface 63 on the interior of the recess 61 is complementary to and engages the curved surface 37 on the first tab 36 when the second housing 50 is fit within the volume 29 of the first housing. The bottom side 60 of the second housing 50 also includes a second recess 65 with a curved surface 67 along an interior of the recess 65. The second recess 65 is positioned along an edge between the bottom side 60 and the second side 58 of the second housing 50 such that it aligns with the second tab 38 on the bottom side 30 of the first housing when the second housing 50 is inserted into the volume 29 of the first housing 20. The width of the second recess 65 is configured to receive the second tab 38 on the bottom side 30 of the first housing. The curved surface 67 on the interior of the recess 65 is complementary to and engages the curved surface 39 on the second tab 38 when the second housing 50 is fit within the volume 29 of the first housing.

The first housing 20 and the second housing 50 include still additional features to facilitate alignment, insertion, and retention of the second housing 50 within the first housing 20. The top side 62 of the second housing 50 includes a first tab 66 and a second tab 68, where each of the first and second tabs 66, 68 protrude along an edge between the top side 62 and the rear side 54 of the second housing. The first tab 66 inserts within a first recess 42 along the top of the volume 29 for the first housing 20, and the second tab 68 inserts within a second recess 44 along the top of the volume 29 for the first housing 20. The top side 62 of the second housing further includes a plurality of tabs 69 protruding from the top side 62 proximate an edge of the top side coincident with the front side 52 of the second housing 50. Each tab 69 engages an edge 46 of the first and second recesses 42, 44 proximate the front side 22 of the first housing 20. Although illustrated with flat surfaces, it is contemplated that the front surface of each tab 69 may be curved, or hooked, to further engage openings along an interior of the first and second recesses 42, 44 to further secure the second housing 50 with the first housing 20.

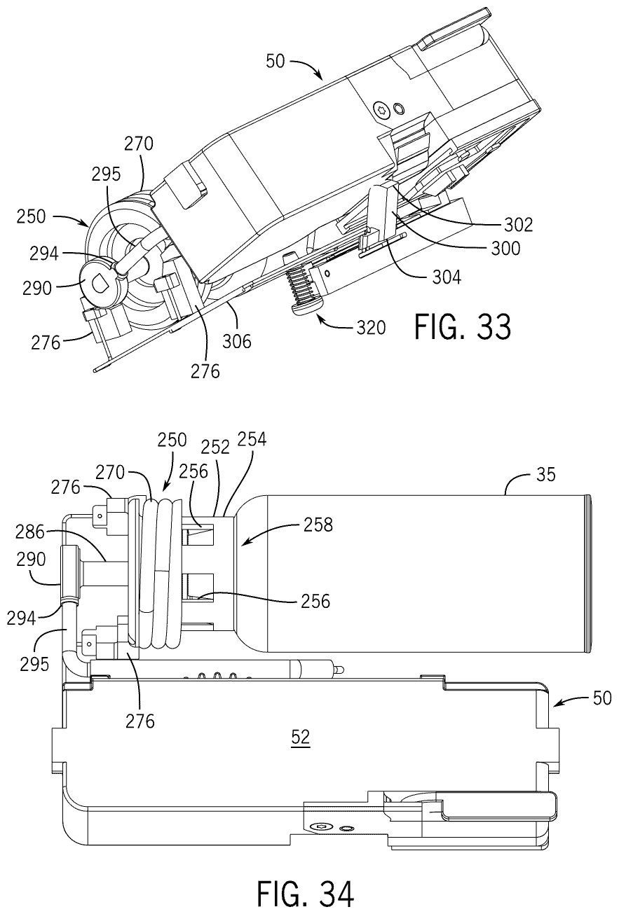

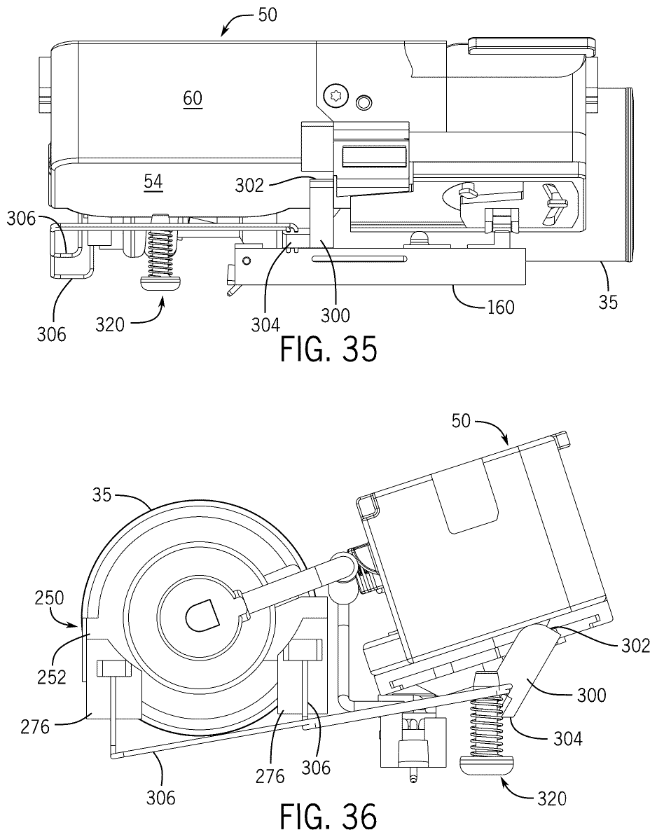

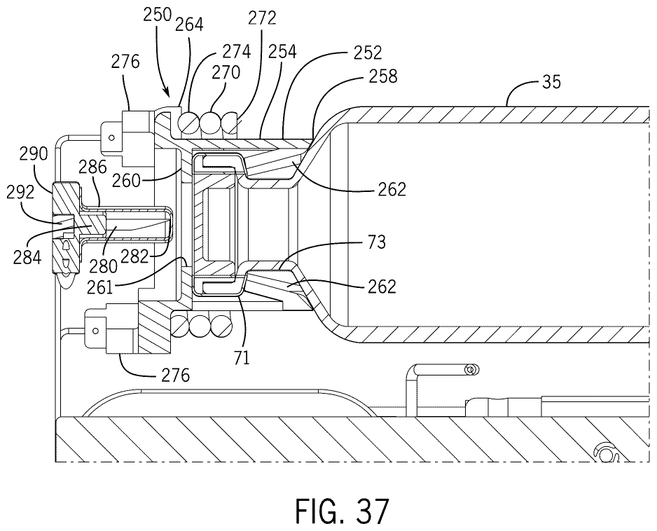

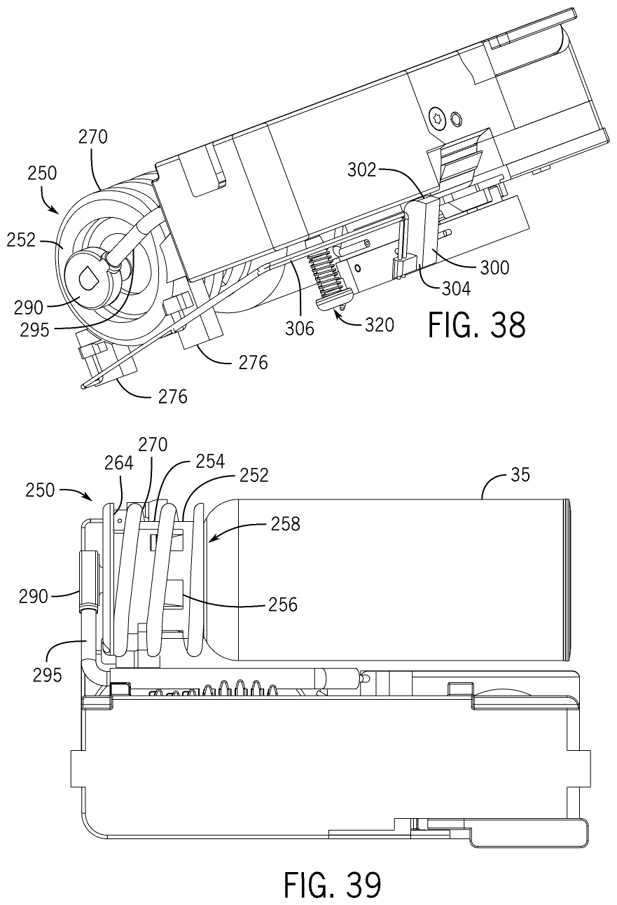

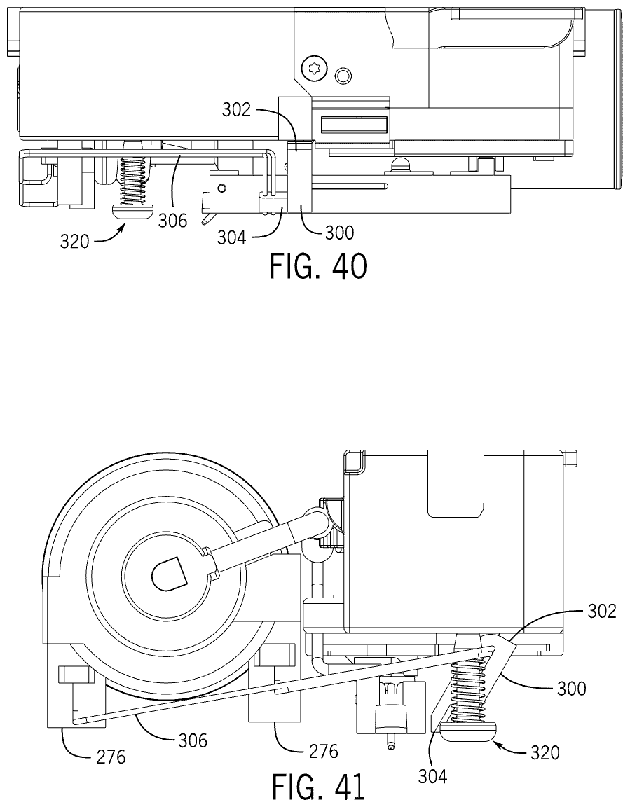

Insertion of the second housing 50 into the volume 29 within the first housing 20 establishes a fluid connection between the cartridge 35 and the cannula assembly 195. With reference next to FIGS. 33-42, a trigger assembly is provided which activates a cartridge adapter assembly 250 to draw the cartridge 35 onto a hollow needle 280 within the cartridge adapter assembly. As best observed in FIG. 37, a cartridge 35 is inserted into a cartridge adapter 252. The illustrated cartridge adapter 252 has a generally cylindrical housing 254. A series of openings 256 are spaced around the periphery of the housing 254. A first end 258 of the cylindrical housing 254 is open and configured to receive the cartridge 35. According to one aspect of the invention, an interior of the housing 254 may include a Luer fitting and be configured to receive a complementary Luer fitting on the cartridge. According to another aspect of the invention, tabs 262 may be provided to retain the cartridge 35 within the cartridge adapter 252. Each tab 262 is positioned within the interior of the housing 254. A first end of each tab 262 is connected proximate the opening on the first end 258 of the housing 254. The tabs 262 extend from the first end 258 toward a second end 260 of the housing 254 and are angled inwards toward the middle of the housing 254. A width between the second ends of each tab is less than a diameter of the mouth 71 of the cartridge 35 to be inserted. Each tab 262 is made from a resilient material such that as the mouth 71 of the cartridge 35 is inserted into the cartridge adapter 252, the tabs 262 are deflected toward an inner periphery of the adapter 252. Once the mouth 71 of the cartridge 35 passes the ends of the tabs 262, the tabs 262 return to their original position and engage a neck 73 of the cartridge 35. Optionally, the neck 73 of the cartridge may also have a diameter greater than a width between the second end of each tab, such that the neck 73 causes some deflection of the tabs 262, and the tabs 262, in turn, provide a holding force against the neck 73 as the material attempts to return to its original, undeflected position. In either case, a diameter of the neck 73 is less than a diameter of the mouth 75 and the tabs 262 return, at least in part, to the original, undeflected position. The tabs 262 are then positioned behind the mouth 71 of the cartridge 35 preventing removal of the cartridge 35 from the cartridge adapter 252.

The cartridge adapter assembly further includes a spring 270 having a first end 272 and a second end 274. The first end 272 of the spring 270 is mounted against a seat within the first housing 20. Optionally, the seat for the first end of the spring 270 may integrally formed as part of the first housing 20. The second end 274 of the spring 270 is mounted against a seat 264 on the cartridge adapter 252. During assembly, the spring 270 is compressed and a pair of spring locks 276 engage the cartridge adapter 252 to hold the cartridge adapter 252 in place with the spring compressed. As will be discussed in more detail below, a trigger mechanism is activated when the second housing 50 is inserted into the volume 29 of the first housing 20 to release the spring locks 276 and to draw the cartridge 35 onto the hollow needle 280.

Prior to insertion of the second housing 50, the hollow needle 280 is located within a sterile housing 286. The sterile housing 286 permits the hollow needle 280 to be sterilized during manufacture and to remain sterile until use by a patient. A first end 282 of the hollow needle 280 is pointed to pierce the sterile housing 286 and a membrane over the mouth of the cartridge 35. A second end 284 of the hollow needle 280 is mounted to a flow connector 290. The flow connector 290 has a first opening in fluid connection with a flow channel through the hollow needle 280 and a second opening in fluid connection with tubing 295 extending from the flow connector 290. The tubing 295, in turn, extends past the pump 330 and to the cannula slide 180 to establish a fluid flow path from the cartridge 35 to the cannula slide 180. The sterile housing 286 is mounted to the flow connector 290 and encloses the hollow needle 280 in a generally cylindrical enclosure extending up beyond the first end 282 of the hollow needle 280.

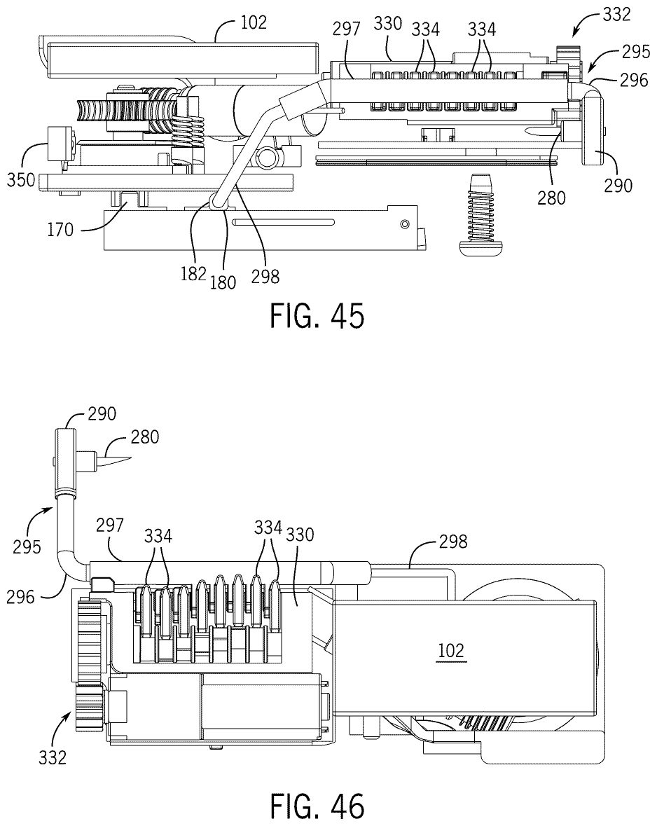

With reference to FIGS. 45 and 46, a pump 330 is provided to draw the medicament from the cartridge 35 and deliver it to the cannula assembly 195. The illustrated pump 330 is a peristaltic pump. A plurality of fingers 334 are reciprocally driven against the tubing 295 in a sequential manner. The fingers 334 may be driven, for example, by a sinusoidal function to sequentially compress and release the tubing 295. Compression of the tubing 295 expels the medicament toward the cannula assembly 195, and releasing the tubing 295 creates a vacuum in the tubing 295 which draws medicament from the cartridge 35. A drive train 332 is provided on the pump which includes, for example, a motor, gears, and a crankshaft with cams to reciprocally drive the fingers 334 against the tubing 295.

The tubing 295 may consist of a combination of rigid tubing and flexible tubing. A first tube segment 296 is rigid and extends from a flow connector 290 toward the peristaltic pump 330. The first tube segment 296 includes a ninety-degree elbow to direct the tubing past the peristaltic pump. A second tube segment 297 connects to the first tube segment 296 and is a compressible tubing configured to engage with the fingers 334 of the peristaltic pump 330. The second tube segment 297 extends for a length of the peristaltic pump and is held in place on either end of the peristaltic pump to ensure that the fingers 334 are able to engage the second tube segment 297. A third tube segment 298 connects to the second end of the second tube segment 297 and extends down to an opening 182 in a cannula slide 180. Within the cannula slide 180, a channel exists between the opening 182 in the cannula slide 180 and an internal opening configured to receive a first end 202 of the cannula 200. The tubing 295, therefore, establishes a fluid flow path between the flow connector 290 and the cannula slide 180.

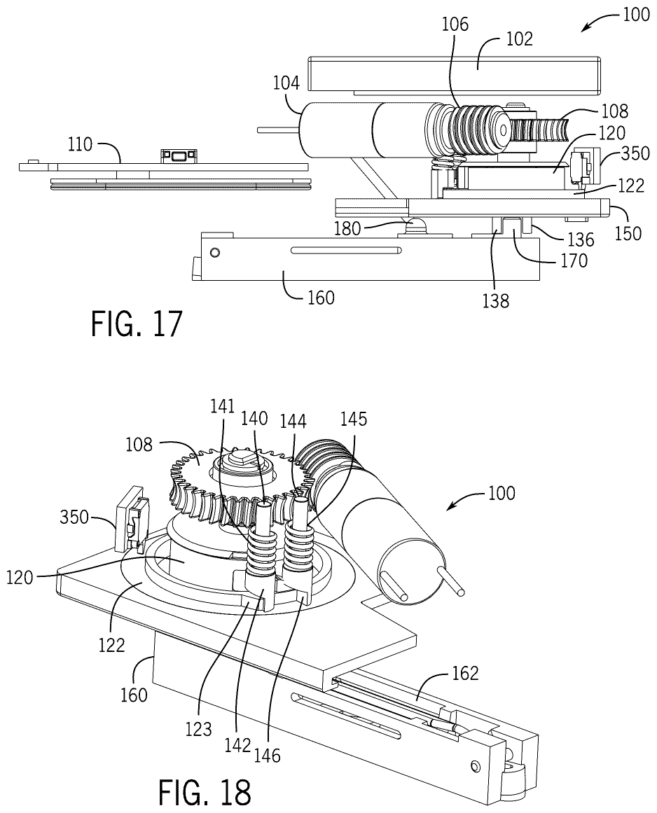

Turning next to FIGS. 16-24, one embodiment of a needle injection mechanism 100 for use with the wearable cartridge pump 10 is illustrated. The needle injection mechanism 100 is divided into two portions, where a first portion of the needle injection mechanism is configured to be disposable, or single-use, and a second portion of the needle injection mechanism is configured to be reusable, or multi-use. The disposable first portion of the needle injection mechanism 100 is mounted within the first housing 20, and the reusable second portion of the needle injection mechanism 100 is mounted within the second housing 50. In this manner, elements of the needle injection mechanism 100, such as the needle 230 and cannula 200, which require sterilization prior to each use, are included in the disposable first portion and may be provided as part of the single-use portion of the wearable cartridge pump 10. Elements of the needle injection mechanism 100, such as the battery 102, winding motor 104, and flywheel 122, used to insert the needle into the patient but which do not require sterilization prior to each use are included in the reusable second portion and provided within the second housing 50 along with the pump 330.

According to the illustrated embodiment, a rechargeable battery 102 is provided to provide power within the reusable portion of the wearable cartridge pump 10, and a motor 104 is provided to drive a worm drive. The worm drive includes a worm gear 106 and a worm wheel 108 to drive axes arranged generally orthogonal to each other. The worm wheel 108 is connected to a clockwork mechanism 120 which includes a torsion spring, and a flywheel 122 is mounted to the clockwork mechanism. The motor 104 is operated to drive the worm gear 106. Rotation of the worm gear 106, in turn, causes rotation of the worm wheel 108. The torsion spring within the clockwork mechanism 120 is connected at one end to an axle mounted between the worm wheel 108 and the clockwork mechanism and at the other end to the flywheel 122. If the flywheel 122 is held in place, then rotation of the worm wheel 108 causes the torsion spring to wind up, increasing the potential energy stored within the clockwork mechanism 120. If the flywheel 122 is released, the torsion spring releases the potential energy stored within the clockwork mechanism by causing rotation of the flywheel 122, as will be discussed in more detail below. The rechargeable battery 102, motor 104, worm drive, clockwork mechanism 120, and flywheel 122 are all reusable components of the wearable cartridge pump 10 and mounted within the second housing 50.

A printed circuit board (PCB) 110 is also mounted within the second housing 50. The PCB 110 includes a control circuit 112 used to control operation of the needle injection mechanism 100. The control circuit 112 may include various integrated circuits such as analog-to-digital (A/D) converters, discrete logic devices, buffers, operational amplifiers, processing devices, memory, and the like. According to the illustrated embodiment, a processor 114 and memory 116 are provided. The processor 114 may be a microprocessor, configured to execute a series of instructions stored in non-volatile memory 116. Optionally, the processor 114 may be an application specific integrated circuit (ASIC), field programmable gate array (FPGA), or other such logic device where a processing component and a memory component may be integrated on a single integrated circuit. The control circuit 112 is configured to receive feedback signals from sensors within the wearable pump assembly 10 and to provide command signals to actuators, such as the motor 104 used to wind the clockwork mechanism 120.

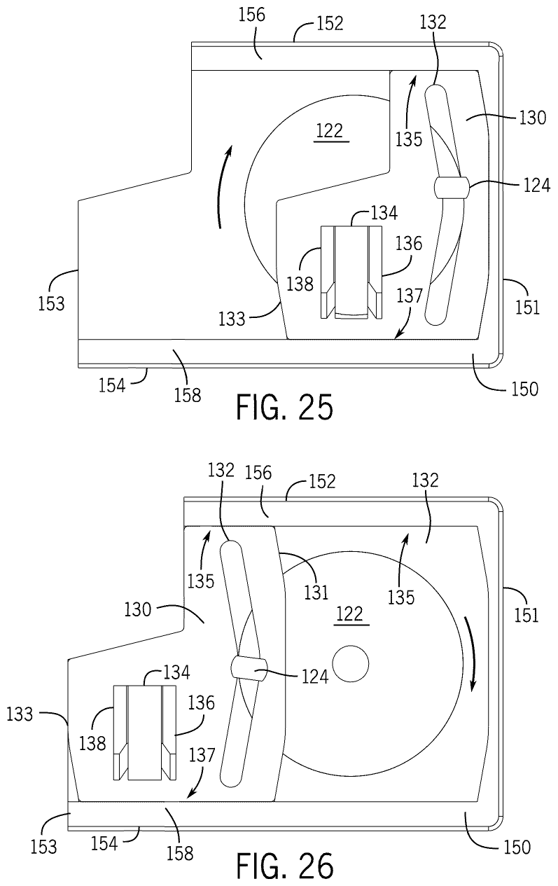

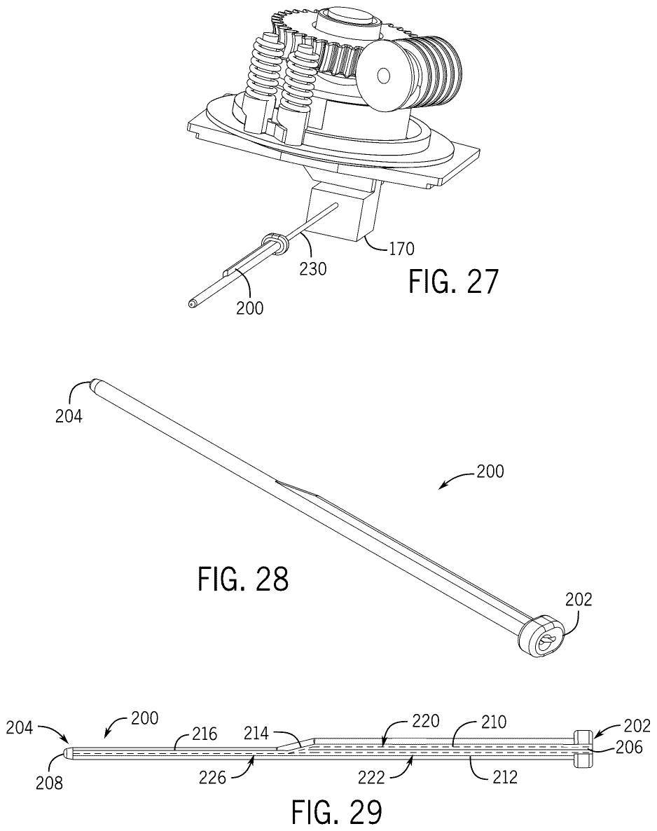

The needle injection mechanism 100 also includes a guide plate 150 mounted to the rear side 54 of the second housing 50. With reference also to FIGS. 25 and 26, the guide plate 150 is an irregular polygon. The guide plate 150 has a first end 151 distal from the side of the wearable cartridge pump 10 from which the needle is inserted into the patient and a second end 153 proximate the side of the wearable cartridge pump from which the needle is inserted into the patient. The guide plate 150 further includes a first rail 156 extending along a first side 152 and a second rail 158 extending along a second side. Each rail 156, 158 defines a channel extending longitudinally along a length of the respective side 152, 154 of the guide plate 150.

A transfer plate 130 is slidably mounted within the guide plate 150. The transfer plate 130 includes a first end 131 distal from the side of the wearable cartridge pump 10 from which the needle is inserted into the patient and a second end 133 proximate the side of the wearable cartridge pump from which the needle is inserted into the patient. The transfer plate 130 further includes a first side 135 and a second side 137 opposite the first side. A width between the first and second sides 135, 137 is sufficient to extend into both of the channels defined by the rails 156, 158 on the guide plate 150. A length of the transfer plate 130 between the first and second ends 131, 133 is less than a length of the guide plate 150 such that the transfer plate 130 is slidably mounted within the channels defined by the rails 156, 158 to move between the first and second ends 151, 153 of the guide plate 150. The transfer plate 130 further includes a V-shaped channel 132 proximate the first end 131 of the transfer plate 130. The V-shaped channel extends generally between the first and second sides 135, 137 of the transfer plate and for a distance less than the width between the rails 156, 158 of the guide plate 150. The peak of the V-shaped channel 132 is pointed toward the first end 131 of the transfer plate, and each leg of the V-shaped channel 132 extends at an angle from the peak toward the second end 133 of the transfer plate 130. The transfer plate 130 also includes a needle slide engagement member region positioned between the V-shaped channel 132 and the second end 133 of the transfer plate 130. According to the illustrated embodiment, the needle slide engagement region includes an opening 134 extending through the transfer plate 130, a first side wall 136 positioned along a side of the opening 134 located closest to the first end 131 of the transfer plate 130, and a second side wall 138 positioned along a side of the opening 134 located closest to the second end 133 of the transfer plate 130. As the second housing 50 is inserted into the first housing 20, the needle slide 170 is received within the opening 134 and between the first and second side walls 136, 138. The opening 134 and side walls 136, 138 engage the needle slide 170 to move the needle slide along a first insertion axis 197 as will be discussed in more detail below.

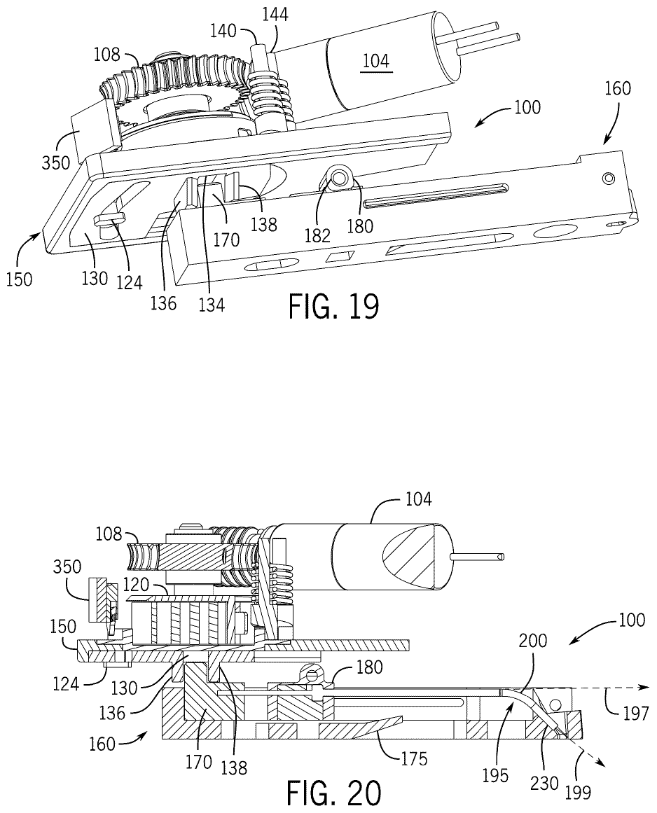

Turning next to FIGS. 19-24, a needle slide assembly 160 according to one embodiment of the invention is illustrated. The needle slide assembly 160 is mounted within the first housing 20 and includes the disposable portion of the needle injection mechanism 100. The needle slide assembly 160 may be secured within the first housing 20 via any number of well-known manufacturing techniques including, but not limited to chemical joining by solvents or adhesives, mechanical joining by vibration welding, ultrasonic welding, hot-plate welding, and the like, or by use of one or more mechanical fasteners. Alternately, at least a portion of the needle slide assembly 160 may be integrally formed with the first housing 20 as part of the molding process. The needle slide assembly 160 includes a channel 162 in which a needle slide 170 and a cannula slide 180 are mounted. As will be discussed in more detail below, the needle slide 170 and cannula slide 180 may be moved from one end of the channel 162 to the other to automatically insert the cannula 200 in a patient for medicament delivery.

In FIGS. 19 and 20, the needle slide 170 and cannula slide 180 are positioned at an initial position. This initial position corresponds to the position at which a patient would receive the wearable cartridge pump 10 from a pharmacy prior to adhering the wearable cartridge pump 10 to the patient's skin. The cannula assembly 195 is in a fully retracted position. A stylet 230 is retracted within the end of the cannula 200, where the stylet 230 is, for example, a hypodermic needle. A first end of the stylet 230 is connected to the needle slide 170. The stylet then extends through a channel, or lumen, in the cannula 200 until a second end of the stylet 230 protrudes from the cannula 200. The second end of the stylet 230 is configured to pierce the skin of the patient as the cannula assembly 195 is inserted into the patient. As will be discussed in more detail below, the needle slide 170 and cannula slide 180 are configured to travel along a first insertion axis 197. A guide mechanism within the needle slide assembly is used to redirect the stylet 230 and cannula 200 from the first insertion axis 197 to a second insertion axis 199. An angle between the first and second insertion axes 197, 199 is less than ninety (90) degrees such that the cannula assembly 195 enters the patient at an angle. According to one embodiment of the invention, the guide mechanism includes a roller and a guide ramp located at the end of the needle slide assembly 160 from which the cannula assembly 195 extends. The stylet 230 is made from a malleable material and the cannula 200 is made from a flexible polymer material. As the cannula assembly 195 is extended, the roller bends the stylet 230 downwards toward the guide ramp. The guide ramp, in turn, causes the cannula assembly 195 to extend along the second insertion axis 199. Optionally, the roller may be a fixed rod having a cylindrical cross-section or have a sloped surface oriented to engage the cannula assembly 195 as the cannula assembly extends.

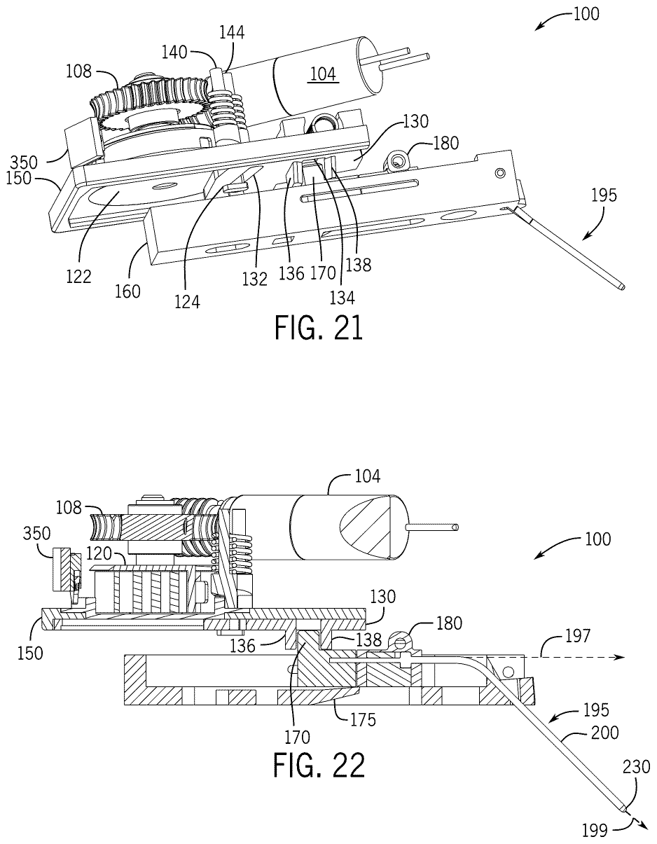

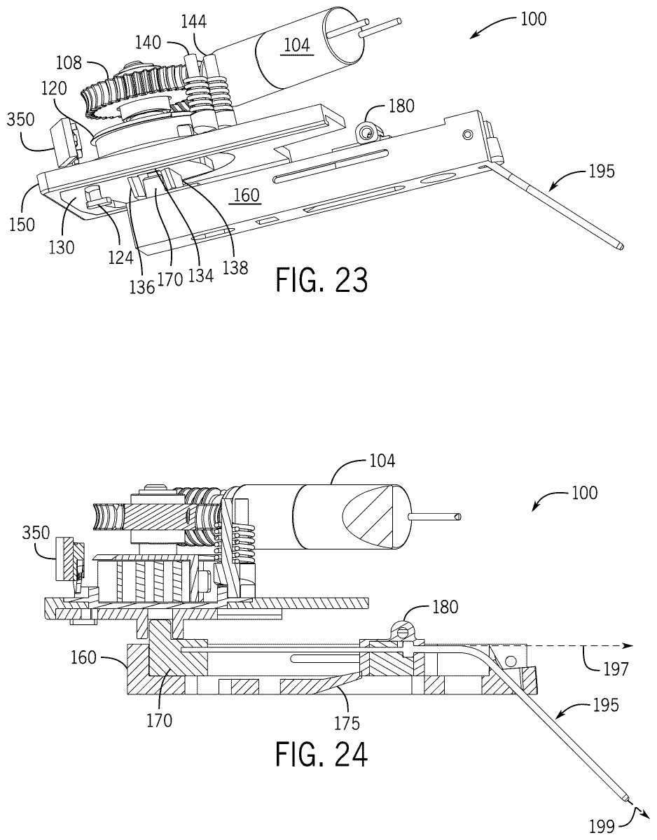

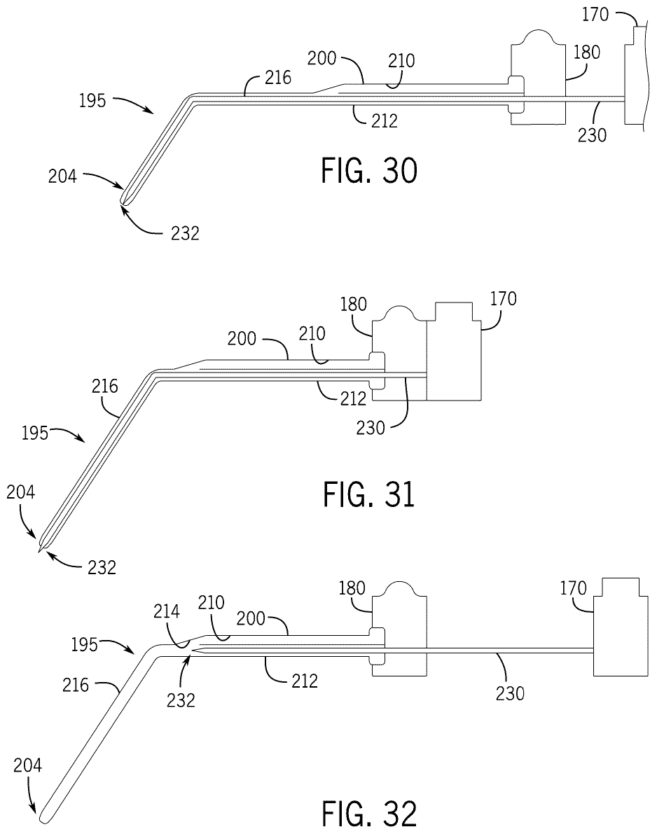

As the cannula assembly 195 extends, the stylet 230 engages the roller and the guide ramp through the cannula 200 causing the stylet 230 to extend along the axes 197, 199 of insertion. The soft outer needle, or cannula, 200 has greater flexibility than the stylet 230 and, therefore, follows the stylet along the axes 197, 199 of insertion. In some embodiments of the invention, the end of the cannula 200 may be closed and the stylet 230 pierces the end of the cannula to open the cannula. Optionally, the end of the cannula 200 may be open and the stylet 230 extends outward through the end of the cannula such that the end of the stylet 230 extends beyond the end of the cannula 200. With reference also to FIGS. 30-31, the end of the stylet 230 extends beyond the end of the cannula such that the stylet first engages the skin of the patient to pierce the skin and to allow the cannula 200 to also be inserted into the skin of the patient. FIG. 21 illustrates the cannula assembly 195 in an intermediate state during insertion of the cannula. The transfer plate 130 is fully extended, causing both the needle slide 170 and the cannula slide 180 to move to the fully extended position. With reference to FIG. 23, the final step of insertion is illustrated. The transfer plate 130 returns to an initial position, causing the needle slide 170 to retract while the cannula slide 180 remains at the extended position. Retracting the needle slide 170 retracts the stylet 230 within the cannula while allowing the cannula 200 to remain extended and inserted within the patient. After this piercing operation, the stiff stylet 230 is retracted leaving only the cannula 200 in place. By removing the stiff stylet 230 in favor of the cannula 200, improved patient comfort may be obtained during delivery of the medicine.

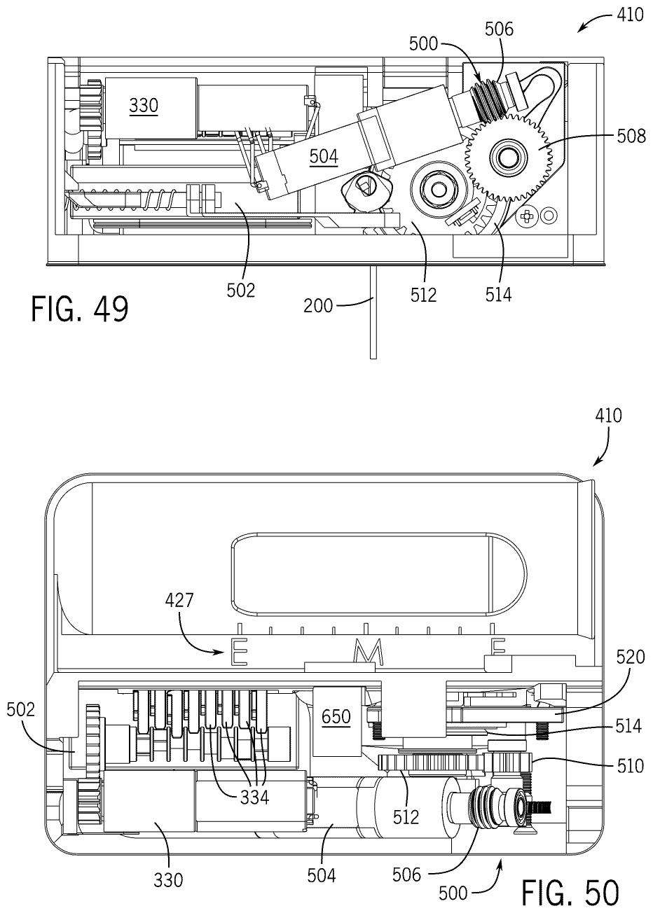

Turning next to FIGS. 48-53, another embodiment of a needle injection mechanism 500 for use with the wearable cartridge pump 410 is illustrated. The needle injection mechanism 500 is divided into two portions, where a first portion of the needle injection mechanism is configured to be disposable, or single-use, and a second portion of the needle injection mechanism is configured to be reusable, or multi-use. The disposable first portion of the needle injection mechanism 500 is mounted within the first housing 420, and the reusable second portion of the needle injection mechanism 500 is mounted within the second housing 450. In this manner, elements of the needle injection mechanism 500, such as the needle 230 and cannula 200, which require sterilization prior to each use, are included in the disposable first portion and may be provided as part of the single-use portion of the wearable cartridge pump 410. Elements of the needle injection mechanism 500, such as the battery 502 and drive motor 504 used to insert the needle 230 into the patient but which do not require sterilization prior to each use are included in the reusable second portion and provided within the second housing 450 along with the pump 330.

According to the illustrated embodiment, a rechargeable battery 502 is provided to provide power within the reusable portion of the wearable cartridge pump 410, and a drive motor 504 is provided to drive the rest of the gear train. A worm gear 506 is mounted on a drive shaft of the drive motor 504 and the worm gear 506 rotates with operation of the motor. The worm gear 506 is operatively engaged with a first spur gear 508, causing rotation of the first spur gear 508 when the worm gear 506 rotates. A second spur gear 510 is mounted coaxially with the first spur gear 508. The second spur gear 510 is operatively engaged with a third spur gear 512 mounted adjacent to the second spur gear. A flywheel 514 is mounted coaxially with the third spur gear 512 such that rotation of the drive motor 504 causes rotation of each of the spur gears 508, 510, 512 and the flywheel 514. The needle injection mechanism 500 also includes a drive arm 520 extending between a first end 522 and a second end 524. The first end 522 of the drive arm 520 is pivotally mounted to the flywheel 514. The second end 524 of the drive arm 520 is pivotally mounted to a first side 532 of a first slide block 530 for a needle slide assembly 525. The drive arm 520 is included within the second housing 450 and is configured to engage the first slide block 530 as the second housing 450 is inserted into the first housing 420. Thus, the drive motor 504 and drive elements up through the drive arm 520 are reusable elements of the wearable cartridge pump 410.

A printed circuit board (PCB) is also mounted within the second housing 450. The PCB includes a control circuit used to control operation of the needle injection mechanism 500. The control circuit may include various integrated circuits such as analog-to-digital (A/D) converters, discrete logic devices, buffers, operational amplifiers, processing devices, memory, and the like. Optionally, the control circuit includes a processor and memory configured to execute a series of instructions stored in non-volatile memory. Optionally, the processor may be a microprocessor or an application specific integrated circuit (ASIC), field programmable gate array (FPGA), or other such logic device where a processing component and a memory component may be integrated on a single integrated circuit. The control circuit is configured to receive feedback signals from sensors within the wearable pump assembly 410 and to provide command signals to actuators, such as the drive motor 504 used to activate the needle injection mechanism 500.

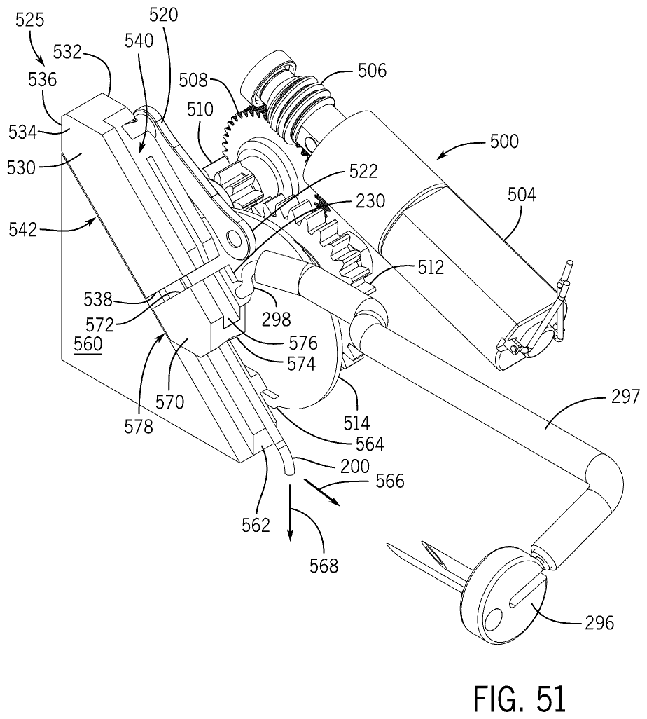

With reference to FIGS. 51-53, a needle slide assembly 525 according to another embodiment of the invention is illustrated. The needle slide assembly 525 includes a first slide block 530 and a second slide block 570 slidably mounted to a slide member 560. The needle slide assembly 525 is mounted within the first housing 420 and includes the disposable portion of the needle injection mechanism 500. The needle slide assembly 525 may be secured within the first housing 420 via any number of well-known manufacturing techniques including, but not limited to, chemical joining by solvents or adhesives, mechanical joining by vibration welding, ultrasonic welding, hot-plate welding, and the like, or by use of one or more mechanical fasteners. Alternately, at least a portion of the needle slide assembly 525, such as the slide member 560, may be integrally formed with the first housing 420 as part of the molding process.

The first slide block 530 includes a second side 534 opposite the first side 532. The first slide block 530 further includes a first end 536 proximate to the pivotal mount of the drive arm 520, and a second end 538 distal from the pivotal mount of the drive arm. The first slide block 530 has an upper surface 540 and a lower surface 542, where the lower surface is configured to engage a guide member. According to the illustrated embodiment, the guide member is a rail 562 of a slide member 560. The second slide block 570 is also mounted on the slide member 560. The second slide block 570 has a first end 572 and a second end 574 opposite the first end 572. The first end 572 of the second slide block 570 is configured to engage the second end 538 of the first slide block 530. The second slide block 570 includes an upper surface 576 and a lower surface 578, where the lower surface is configured to engage the rail 562 of the slide member 560.

In FIG. 51, the needle slide assembly 525 is positioned at an initial position. This initial position corresponds to the position at which a patient would receive the wearable cartridge pump 410 from a pharmacy prior to adhering the wearable cartridge pump 410 to the patient's skin. The first slide block 530 and the second slide block 570 are in a fully retracted position. The stylet 230 is retracted within the end of the t 200, where the stylet 230 is, for example, a hypodermic needle. A first end of the stylet 230 is connected to the first slide block 530. The stylet then extends through a channel in the cannula 200 until a second end of the stylet 230 is proximate to or protrudes from the cannula 200. The second end of the stylet 230 is configured to pierce the skin of the patient as the needle slide assembly 525 extends. The first slide block 530 and the second slide block 570 are configured to travel along the rail 562 of the slide member 560 in the direction of a first insertion axis 566. A guide mechanism associated with the needle slide assembly 525 is used to redirect the stylet 230 and cannula 200 from the first insertion axis 566 to a second insertion axis 568. According to one embodiment of the invention, the guide mechanism includes a roller and/or a guide ramp located at the end of the needle slide assembly 525 from which the cannula assembly extends. The stylet 230 is made from a malleable material and the cannula 200 is made from a flexible polymer material. As the cannula assembly is extended, guide mechanism bends the stylet 230 toward an opening in the rear side 424 of the first housing 420 and toward the patient's skin. The soft outer needle, or cannula, 200 has greater flexibility than the stylet 230 and, therefore, follows the stylet along the axes 566, 568 of insertion. In some embodiments of the invention, the end of the cannula 200 may be closed and the stylet 230 pierces the end of the cannula to open the cannula. Optionally, the end of the cannula 200 may be open and the stylet 230 extends outward through the end of the cannula such that the end of the stylet 230 extends beyond the end of the cannula 200. As the stylet 230 extends through the opening in the rear side 424 of the first housing 420, the end of the stylet 230 extends beyond the end of the cannula such that the stylet first engages the skin of the patient to pierce the skin and to allow the cannula 200 to also be inserted into the skin of the patient.

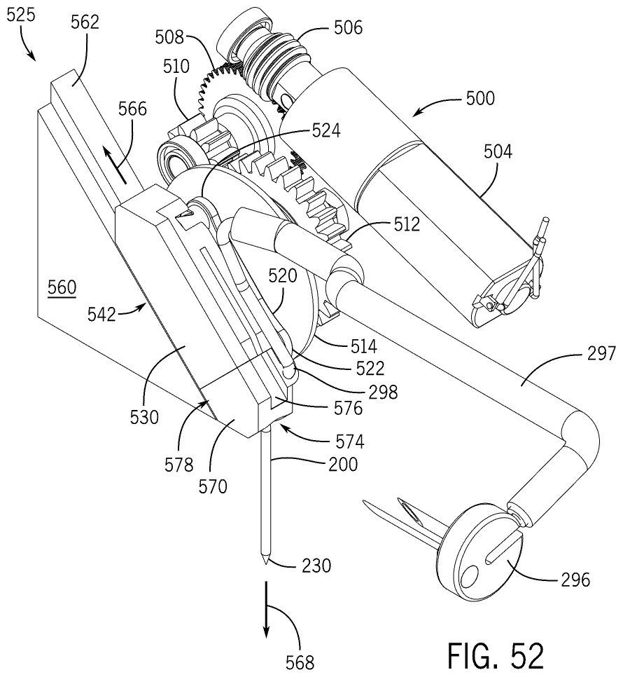

In FIG. 52, the needle slide assembly 525 is positioned at an intermediate state during insertion of the cannula. The flywheel 514 has rotated one hundred eighty degrees. The first end 522 of the drive arm 520 is pivotally mounted to the flywheel 514 and follows the flywheel throughout the rotation. The drive arm 520 is in an extended state, and the second end 524 of the drive arm 520 has caused the first slide block 530 to move along the rail 562 of the slide member 560. As the first slide block 530 moves along the rail 562, the second end 538 of the first slide block 530 engages the first end 572 of the second slide block 570, pushing the second slide block 570 along the rail 562. The stylet 230 slidably moves through the lumen of the cannula 200 until the second end of the stylet 230 extends beyond the second end of the cannula 200. The second end of the stylet 230 is configured to pierce the skin of the patient as the cannula 200 and stylet 230 are inserted into the patient.

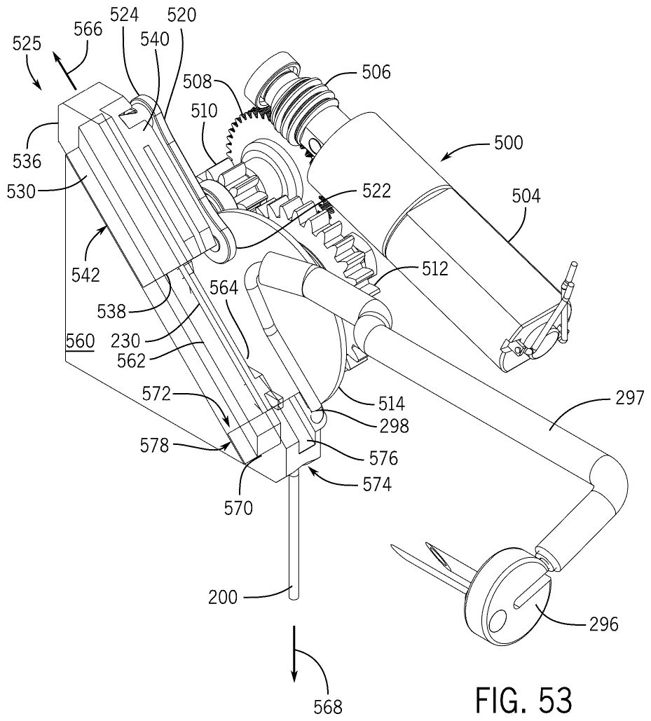

In FIG. 53, the needle slide assembly 525 is at a final state and has completed insertion of the cannula 200 and retraction of the stylet 230 such that fluid may be delivered from the reservoir to the cannula 200. The flywheel 514 continues rotating from the position illustrated in FIG. 52 until a full revolution is completed. The drive arm 520 is again in a retracted state, and as the first end 522 of the drive arm 520 moves with the flywheel 514, the second end 524 of the drive arm 520 pushes the first slide block 530 back up the guide rail 562 to the retracted position. As viewed in FIG. 51, a retention tab 564 is provided along one side of the rail 562. The retention tab 564 is made of a resilient material and, in an initial position, is located within a path of travel for the second slide block 570. As the second slide block 570 travels along the rail 562, the second slide block 570 deflects the retention tab 564 from the initial position to a deflected position. As the first slide block 530 pushes the second slide block 570 to the fully extended position, the second slide block 570 travels beyond the retention tab 564. As the first slide block 530 returns to its first position, it clears the retention tab 564, allowing the retention tab to return to its initial position. With the retention tab 564 in the initial position and the second slide block 570 in the extended position, the retention tab 564 interferes with motion of and holds the second slide block 570 in the extended position.

In operation, the wearable cartridge pump 10 provides for delivery of a medicament to a patient with a reusable pump assembly. According to one aspect of the invention, a patient, P, will receive both the first housing 20 and the second housing 50 along with a cartridge 35 of prescribed medicament as an initial prescription from a doctor. After the initial fill of the prescription, a patient may receive just a first housing 20 and a cartridge 35 of prescribed medicament as refills while reusing the second housing 50. In one aspect of the invention, the pharmacist or drug manufacturer may insert the cartridge 35 into the first housing 20 prior to delivery to the patient, such that the patient only inserts the second housing 50 into the first housing 20. In another aspect of the invention, the cartridge 35 and the first housing 20 may be provided to the patient separately such that the patient first inserts the cartridge 35 into the first housing 20 and then inserts the second housing 50 into the first housing 20.

With reference again to FIGS. 33-42, insertion of the second housing 50 into the first housing 20 activates a trigger assembly within the first housing 20. An actuator 300 extends upward from the first housing 20 into the volume 29 of the first housing 20. According to the illustrated embodiment, the actuator 300 is a slide arm which moves between a first position and a second position. A first end 302 of the slide arm 300 extends into the volume 29 of the first housing 20 when the slide arm is in a first position and creates an interference fit for the second housing 50 as it is inserted into the volume 29. Insertion of the second housing 50 presses down on the slide arm 300 causing the slide arm to move from the first position to a second position. In the second position, the slide arm 300 is recessed below the volume 29 and within the first housing 20. Although illustrated as a sliding member, the actuator 300 may be pivotally mounted or mounted in any other suitable manner to transition between a first position and a second position as a result of insertion of the second housing 50 into the volume 29 of the first housing 20.

Movement of the actuator 300 between the first and second positions causes the spring locks 276 to disengage from the cartridge adapter. A second end 304 of the slide arm 300 has a pair of linkages 306 connected thereto. A first linkage 306 extends between the slide arm 300 and the slide lock 276 on one side of the cartridge adapter 252, and a second linkage 306 extends between the slide arm 300 and the slide lock 276 on the other side of the cartridge adapter 252. Movement of the slide arm 300 between the first and second positions, causes the linkages 306 to similarly transition between a first and second position. According to the illustrated embodiment, each linkage 306 is a rigid arm with a first end mounted to the slide arm 300 and a second end mounted to one of the slide locks 276 in a vertical orientation. Translation of the slide arm 300 between the first and second positions causes the second end of each linkage 306 to travel downward in the vertical direction, pulling the slide lock 276 along with the linkage 306. With the slide arm 300 in the second position, the linkages 306 have caused their respective slide lock 276 to completely disengage from the cartridge adapter, releasing the holding force on the spring 270 within the cartridge adapter assembly.

Releasing the holding force on the spring 270 in the cartridge adapter assembly 250 permits the spring 270 to expand, drawing the cartridge 35 into the first housing 20 and onto the hollow needle 280 within the cartridge adapter assembly 250. As noted above, the first end 272 of the spring 270 is mounted against a seat within the first housing 20, and the second end 274 of the spring 270 is mounted against a seat 264 on the cartridge adapter 252. The seat within the first housing 20 is fixed with respect to the housing, while the seat 264 on the cartridge adapter 252 can move. The force applied by the spring is sufficient to draw the cartridge adapter 252 toward the side of the wearable cartridge pump 10 and to pull the cartridge 35 further into the first housing 20.

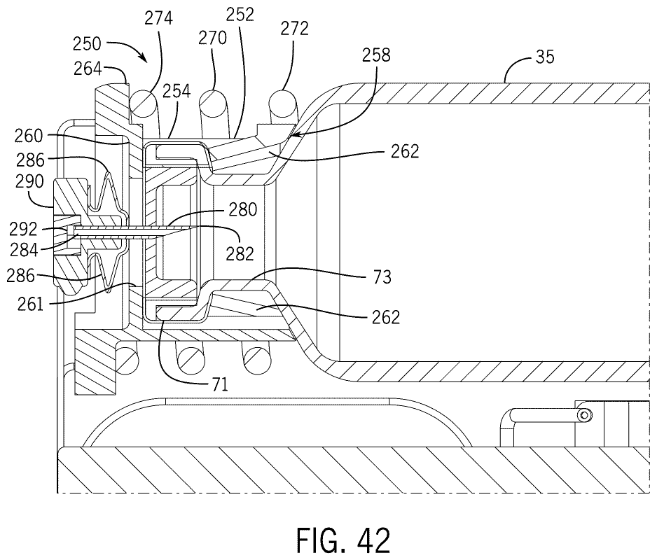

As best illustrated in FIGS. 37 and 42, the sterile housing 286 will engage the mouth 71 of the cartridge 35 as the spring 270 pulls the cartridge adapter 252 between the initial position and a second position. An opening 61 is present in the second end 260 of the cartridge adapter 252. The sterile housing 286 for the hollow needle 280 will pass through the opening 261 and engage the mouth 71 of the cartridge 35. According to one embodiment of the invention, the cartridge 35 includes a self-sealing membrane over the mouth 71. The self-sealing membrane allows a needle 289 to pass through to the interior of the cartridge 35 while creating a seal around the needle to prevent fluid from leaking out of the cartridge into an interior of the first housing 20. When the sterile housing 286 engages the membrane over the mouth 71 of the cartridge 35, the sterile housing 286 will begin to compress. The sterile housing 286 is made from an elastomeric material such that is stands upright around the hollow needle 280 prior to engagement, but as the cartridge adapter 252 is pulled toward the needle 280 by the spring 270 expansion and as the sterile housing 286 engages the membrane, the sterile housing 286 compresses toward the first end 282 of the hollow needle. The first end 282 of the hollow needle 280 is pointed such that it pierces the sterile housing 286 and the membrane on the cartridge. As seen in FIG. 42, the sterile housing 286 slides down the hollow needle 280 and is compressed into a void which remains after the cartridge adapter is fully extended. The first end 282 of the hollow needle 280 is then located within the cartridge 35 to receive the medicament.

Automatic activation of the cartridge adapter assembly 250 provides several benefits to the patient. As previously discussed, the cartridge may be inserted into the first housing 20 by the pharmacist. Because the cartridge is not pierced at that time, the first housing 20 and the cartridge 35 may be stored for an extended period of time. The medicament within the cartridge 35 remains in its sterile enclosure until the second housing 50 is inserted into the first housing at which time, the cartridge 35 will be drawn onto the hollow needle 280 and a fluid flow path established. Further, the automatic activation of the cartridge adapter assembly 250 does not require a patient to insert a cartridge onto the hollow needle 280. If a patient inserts the cartridge 35 into the first housing 20, it need only be inserted until it is retained within the cartridge adapter 252. Subsequent insertion of the second housing 50 will ensure proper establishment of the fluid flow path.

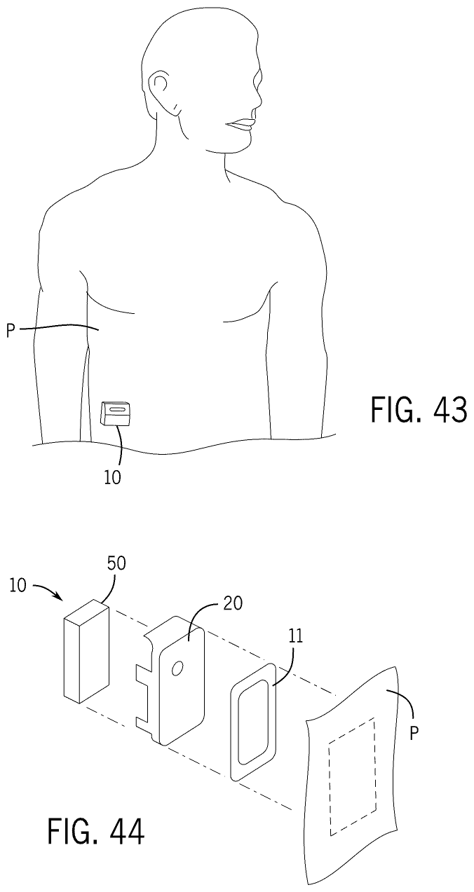

With both housings 20, 50 connected, the patient, P, adheres the wearable cartridge pump 10 to an anatomical region on the patient at which the medicament is to be delivered. With reference to FIGS. 43 and 44, a double-sided adhesive sheet 11 may be provided and adhered to a rear surface of the first housing 20. According to the illustrated embodiment, the adhesive sheet 11 is provided as a peripheral ring around the rear of the first housing 20. Optionally, the adhesive sheet 11 may be of any suitable geometry to provide a broad area of attachment to the patient's skin. The adhesive of the patient-side of the double-sided adhesive sheet 11 may be protected by a release liner to be removed by the patient prior to adhering the wearable cartridge pump 10 to his or her skin.

A skin sensor 320 may be provided on the rear side of the wearable cartridge pump 10. The skin sensor 320 generates a signal to the control circuit 112 in the wearable cartridge pump 10 that the pump is attached to the skin of the patient, P, and is ready to be used. Further, the needle injection mechanism 100 described below may not operate until the skin sensor 320 indicates that the wearable cartridge pump 10 is attached to the skin as a safety precaution. The patient, P, may press a start button or switch which communicates with the control circuit 112 to send a signal to the needle injection mechanism 100 to begin the injection process as described further below.

According to another embodiment of the invention, the container for the medicament may be a vial or a drug bag. An alternate housing may be utilized to support the different containers. A pouch and a neck strap may be provided into which the vial and drug bag are inserted. An over-the-shoulder bag, with a zippered top, flap, open pouch or the like may be provided to receive the container for the fluid. Each container serves as a reservoir for the medicament to be delivered to the patient. According to still another aspect of the invention, the container may be sized to fit within a pocket of the patient. For each container, a suitable connection point is provided to couple the container to the tubing 295. The needle injection mechanism is positioned on the body of the patient, and tubing 295 establishes a fluid flow path from the container to the needle injection mechanism. Any of the disclosed needle injection mechanisms may utilized in combination with the different containers to provide a drug delivery system to the patient.

After adhering the wearable cartridge pump 10 to the desired anatomical region or positioning the container within the corresponding holder, a patient may activate the needle injection mechanism 100 or 500, for example, by pressing a button on the wearable cartridge pump 10. The button sends a signal to the control circuit 112 which controls operation of the needle injection mechanism 100 or 500. With reference to FIG. 18, a first embodiment of the needle injection mechanism 100 includes a first stop 140 and a second stop 144. The first stop 140 has a first spring 141 and a first engagement surface 142. The second stop 144 has a second spring 145 and a second engagement surface 146. When each of the first and second stops 140, 144 are de-energized, the respective spring 141, 145 applies a force in the downward direction such that the first and second engagement surfaces 142, 146 are adjacent an upper surface of the flywheel 122. After winding the clockwork mechanism 120, the potential energy within the torsion spring contained by the clockwork mechanism 120 applies a biasing force on the flywheel 122 to rotate toward the first stop 140. A boss 123 on the upper surface of the flywheel 122 engages the first engagement surface 142 of the first stop 140, preventing further rotation of the flywheel 122. When the control circuit 112 receives the signal to activate the needle injection mechanism, the control circuit 112 first activates an actuator in the first stop 140. The actuator may be, for example, a solenoid which causes the first engagement surface 142 to raise up and to compress the first spring 141. The first engagement surface 142 raises a sufficient distance such that the first engagement surface 142 no longer interferes with the boss 123 on the flywheel 122. The torsion spring within the clockwork mechanism 120, therefore, causes the flywheel 122 to begin rotation. The flywheel 122 will rotate until the boss 123 on the flywheel engages the second engagement surface 146 on the second stop 144. After the boss 123 on the flywheel engages the second engagement surface 146, the control circuit deactivates the actuator in the first stop 140. The first spring 141 causes the first stop 140 to lower back to the surface of the flywheel 122. The control circuit 112 next activates an actuator in the second stop 144. The actuator may be, for example, a solenoid which causes the second engagement surface 146 to raise up and to compress the second spring 145. The second engagement surface 146 raises a sufficient distance such that the second engagement surface 146 no longer interferes with the boss 123 on the flywheel 122. The torsion spring within the clockwork mechanism 120, therefore, causes the flywheel 122 to begin rotation. The flywheel 122 will complete a rotation and continue rotating until the boss 123 on the flywheel again engages the first engagement surface 142 on the first stop 140. After the boss 123 on the flywheel passes the second engagement surface 146, the control circuit deactivates the actuator in the second stop 144. The second spring 145 causes the second stop 144 to lower back to the surface of the flywheel 122. The sequence of raising and lowering the first and second stops 140, 144 provides for controlled rotation of the flywheel 122 through one revolution.

A sensor 350 is provided to detect rotation of the flywheel 122. The sensor may be, for example, a microswitch which is activated as the boss 123 passes the sensor 350. Optionally, an optical sensor may emit radio frequency (RF) radiation in the visible spectrum, infrared spectrum, ultraviolet spectrum, or any suitable spectrum. The RF radiation is reflected from the boss 123 as the boss passes the sensor 350. The sensor 350 detects the reflected radiation to determine that the flywheel 122 has rotated past the sensor. According to still another aspect of the invention, the sensor 350 may be a magneto-receptive sensor, such as a Hall effect sensor. A magnet may be mounted on the flywheel 122 and the sensor 350 detects the magnetic field as the magnet passes the sensor 350. The sensor 350 generates a feedback signal corresponding to rotation of the flywheel 122 and transmits the feedback signal to the control circuit 112.

Rotation of the flywheel 122 through one revolution is used to inject the cannula assembly 195 into a patient. With reference again to FIGS. 19-20, the needle injection mechanism is illustrated in an initial position. In this initial position, a second button on the wearable cartridge pump 10 may be pressed to wind the clockwork mechanism 120. Because the first stop 140 is holding the flywheel 122 in place, rotation of the worm wheel 108 by the motor 104 will wind the torsion spring within the clockwork mechanism 120. According to one aspect of the invention, the clockwork mechanism 120 may be wound with the second housing 50 detached from a first housing 20. Each of the components are mounted in or on the second housing 50 and permits resetting the clockwork mechanism 120 between uses. An indicator, such as a light-emitting diode (LED) may be provided to indicate whether the clockwork mechanism 120 is wound. Alternately, the clockwork mechanism 120 may be wound after inserting the second housing 50 into a new first housing 20. Because the first stop 140 prevents rotation of the flywheel 122, the needle slide assembly 160 is not activated while winding the clockwork mechanism 120.

During the first half of one rotation of the flywheel 122, the flywheel transitions from an initial, retracted position shown in FIG. 25 to a second, extended position shown in FIG. 26. The flywheel 122 includes a boss 124 protruding from a lower surface of the flywheel to engage the V-shaped channel 132 in the transfer plate 130 during rotation of the flywheel. In the illustrated embodiment, the flywheel 122 rotates in a clock-wise direction as indicated by the arrows in FIGS. 25 and 26. During the first ninety degrees of rotation, the boss 124 will slide within the channel 132 toward a first end of the channel, causing the transfer plate 130 to begin extending within the guide plate 150. As the flywheel 122 rotates between ninety and one hundred eighty degrees, the boss 124 slides back toward the center of the V-shaped channel 132, arriving at the position shown in FIG. 26.

This first half of rotation of the flywheel 122 will also extend the needle slide assembly 160 from the position shown in FIGS. 19 and 20 to the position shown in FIGS. 21 and 22. As the transfer plate 130 moves forward, the needle slide 170, retained between the first and second side walls 136, 138 of the opening 134, engages the cannula slide 180 pushing the cannula slide 180 forward as well. The rotation of the flywheel 122 causes the transfer plate 130 to slide forward and, in turn, the transfer plate moves the needle slide 170 and the cannula slide 180 forward to the extended position. A retention tab 175 is present along the floor of the channel within the needle slide assembly 160. With the needle slide 170 in the extended position, the retention tab 175 is deflected downward below the floor of the channel. Extension of the needle slide 170 and of the cannula slide 180 cause the cannula assembly 195 to extend. As the cannula assembly 195 extends, the sharp end 232 of the stylet 230 pierces the skin of the patient, permitting both the stylet 230 and the cannula 200 to be inserted subcutaneously within the patient.

During the second half of rotation of the flywheel 122, the flywheel transitions from the second, extended position shown in FIG. 26 back to the initial, retracted position shown in FIG. 25. The flywheel 122 continues rotating in the clock-wise direction indicated. As the flywheel 122 rotates between one hundred eighty degrees and two hundred seventy degrees, the boss 124, protruding from the flywheel 122, will slide within the channel 132 toward a second end of the channel, causing the transfer plate 130 to begin retracting within the guide plate 150. As the flywheel 122 rotates from two hundred seventy degrees back to the original starting position, the boss 124 slides back toward the center of the V-shaped channel 132, arriving at the position shown in FIG. 25.

The second half of rotation of the flywheel 122 will transition the needle slide assembly 160 from the position shown in FIGS. 21 and 22 to the position shown in FIGS. 23 and 24. As the transfer plate 130 moves back, the needle slide 170, retained between the first and second side walls 136, 138 of the opening 134 is pulled away from the cannula slide 180. Further, the needle slide 170 moves off the retention tab 175 along the bottom of the channel in the needle slide assembly 160. The retention tab 175 is made of a resilient material such that it may be deflected away from its original position by the needle slide 170 but returns to its original position when the needle slide 170 is retracted. As best seen in FIG. 24, the retention tab 175 raises up into the channel of the needle slide assembly 160 to hold the cannula slide 180 in the extended position. The transfer plate 130 will pull the needle slide back to its original position, retracting the stylet 230 within the cannula assembly 195 but leaving the cannula 200 inserted subcutaneously within the patient.

Turning next to FIGS. 30-32, operation of the cannula assembly 195 is further illustrated during the insertion process. In FIG. 30, the stylet 230 and the cannula 200 are both in the retracted position. A sharp end 232 of the stylet is shown extending beyond a second end 204 of the cannula 200. Optionally, the sharp end 232 of the stylet may initially be positioned within the cannula 200 and may pierce the second end 204 of the cannula during insertion into the patient. In FIG. 31, the stylet 230 and the cannula 200 are both extended to complete the subcutaneous insertion of the cannula assembly 195 in the patient. In FIG. 32, the cannula 200 remains extended while the stylet 230 is retracted. As further seen in FIG. 32, the stylet 230 is retracted beyond an opening 214 internal to the cannula 200 which provides for improved fluid flow through the cannula.

Turning next to FIGS. 28 and 29, the cannula 200 may be configured to include parallel flow paths 220, 222 for at least a portion of the length of the cannula. The cannula 200 has a first end 202 configured for connection to the cannula slide 180 and a second end 204 configured for subcutaneous insertion into the patient. A first opening 206 is present in the first end 202 of the cannula 200, and a second opening 208 is present in the second end 204 of the cannula. At the first end 202 of the cannula 200, a first channel 210 and a second channel 212 extend in parallel to each other for a first portion of the cannula. At the internal opening 214 within the cannula 200, the first channel 210 and the second channel 212 merge to a combined channel 216. The combined channel 216 extends from the internal opening 214 to the second opening 208 of the cannula. Each of the first channel 210 and the second channel 212 are in fluid communication with the cannula slide 180 to receive medicament passing through the cannula slide. The first fluid flow path 220 extends along the first channel 210, and the second fluid flow path 222 extends along the second channel 212. The first and second flow paths 220, 222 combine at the internal opening to form a combined flow path 226, where the combined flow path 226 extends for the length of the combined channel 216.

The parallel flow paths 220, 222 provide for improved flow of the medicament to the patient. As shown in FIG. 32, when the stylet 230 is retracted and the cannula 200 is subcutaneously inserted into the patient, the second end 232 of the stylet is retracted beyond the internal opening 214 providing for a clear flow path from the cannula slide 180 to the second end 204 of the cannula 200. In prior cannula assemblies, a single flow path exists between the first and second ends 202, 204 of the cannula such that the stylet 230 remains within the flow path. Although the stylet 230 may be a hollow needle, permitting some fluid flow through the needle as well as additional fluid flow around the needle, the stylet 230 obstructs at least a portion of the fluid flow through the flow path. In order to achieve a desired flow rate through a channel in which the stylet 230 is present, the diameter of the cannula 200 must be sufficient to allow fluid to flow around the stylet 230. The increased diameter cannula 200 must also be inserted into the patient, increasing potential discomfort to the patient.

In contrast, the cannula 200 illustrated in FIG. 28 provides one flow path 222 which still receives the stylet 230. However, another flow path, including the combination of the first flow path 220 and the combined flow path 226 provides a clear flow path between the cannula slide 180 and the patient. The diameter of the cannula 200 is greater where the two flow paths 220, 222 are present, but the diameter is reduced at the combined flow path 226 to the second end 203 of the cannula 200 which is inserted into the patient. The reduced diameter of the cannula 200 to achieve the same flow rate provides for improved comfort to the patient.

In addition to improved comfort for the patient, the illustrated cannula 200 may also provide for improved delivery of the medicament from the cartridge 35 to the patient. Different medicaments have different viscosities. For medicaments with greater viscosity, an increased diameter flow path may be required. Providing the first flow path 220 in parallel to the channel 212 in which the stylet 230 is present, increases the diameter of the flow path along the first portion of the cannula 200. If the medicament were required to flow through a hollow stylet 230 and/or around the outer periphery of the hollow stylet 230, the overall diameter of the flow path would be substantially reduced. Therefore, medicaments of higher viscosity may be delivered through the cannula 200 with the parallel flow path 220 than with a single continuous flow path between the two ends. Similarly, doses of a medicament requiring an increased flow rate may be provided through the first flow path 220. If the medicament were required to flow through a hollow stylet 230 and/or around the outer periphery of the hollow stylet 230, the interference of the hollow stylet 230 would create turbulence in the flow path, reducing the maximum potential flow rate. Delivering medicament through a clear first flow path 220 reduces interference and allows for an increased flow rate. Therefore, a medicament may be delivered at an increased flow rate with the parallel flow path 220 than with a s ingle continuous flow path between the two ends.

According to another aspect of the invention, the first opening 206 may include a self-healing membrane over the second channel 212. Thus, the stylet 230 may be inserted through the self-healing membrane for use during insertion of the cannula assembly 195. After insertion, the stylet 230 is retracted beyond the internal opening 214 within the cannula and a single flow path is defined from the first opening 206 to the second opening 208 via the first flow path 220 and the combined flow path 226.

With reference again to FIGS. 51-53, a second embodiment of the needle injection mechanism 500 includes a motor 504 used to drive the drive train. The patient may activate the needle injection mechanism 500, for example, by pressing a button on the wearable cartridge pump 410. The button sends a signal to the control circuit which controls operation of the needle injection mechanism 500. When the control circuit receives the signal to activate the needle injection mechanism, the control circuit enables the motor 504. The motor 504 is a high-speed, low-torque motor. The drive train includes multiple gears 506, 508, 510, 512 to reduce the speed and increase the torque applied to the needle injection mechanism 500 to facilitate insertion of the cannula 200 into a patient. A worm gear 506 is mounted on a drive shaft extending from the motor 504 and rotates with rotation of the motor. Each of the additional gears 508, 510, 512 are sequentially driven from the initial worm gear 506, and the flywheel 514 is coaxially mounted with the final additional gear 512 such that the flywheel 514 rotates when the drive motor 504 rotates.

Rotation of the flywheel 514 through one revolution is used to inject the cannula 200 into the patient. With reference again to FIG. 51, the flywheel 514, drive arm 520, first slide block 530, and second slide block 570 are each illustrated in an initial position. During the first half of a revolution, the flywheel 514 is driven from this initial, retracted position, to a second, extended position, shown in FIG. 52. Rotation of the flywheel 514 causes the drive arm 520 to pull the first slide block 530 and the second slide block 570 along the rail 562, as discussed above. Initially, the second end of the stylet 230 may be positioned within the lumen of the cannula 200. During the initial motion of the first slide block 530, the stylet slides within the lumen of the cannula 200 such that the second end of the stylet 230 extends beyond the second end of the cannula 200 such that the stylet 230 is able to pierce the skin of the patient.

When the first slide block 530 fully engages the second slide block 570, the two slide blocks move in tandem along the rail 562. The first end of the stylet 230 is mounted to the first slide block 530, and the first end of the cannula 200 is mounted to the second slide block 570. As the first and second slide blocks 530, 570 move along the rail 562, the stylet 230 and cannula 200 similar move along the first insertion axis 566 to be inserted into the patient. As the stylet 230 and cannula 200 reach the end of the slide member 560 they are deflected from the first insertion axis 566 to the second insertion axis 568 for insertion into the patient. The stylet 230 and cannula 200 exit the opening in the bottom of the needle injection mechanism 500 and enter the patient. In FIG. 52, the stylet 230 and the cannula 200 are illustrated in the fully extended position.

During the second half of the revolution of the flywheel 514, the drive arm 520 moves back to the initial position, pushing the first slide block 530 back up the rail 562, and the retention tab 564 holds the second slide block 570 at the extended position. Because the stylet 230 is mounted to the first slide block 530, the stylet retracts from within the lumen of the cannula 200 while the cannula 200 remains inserted subcutaneously within the patient.

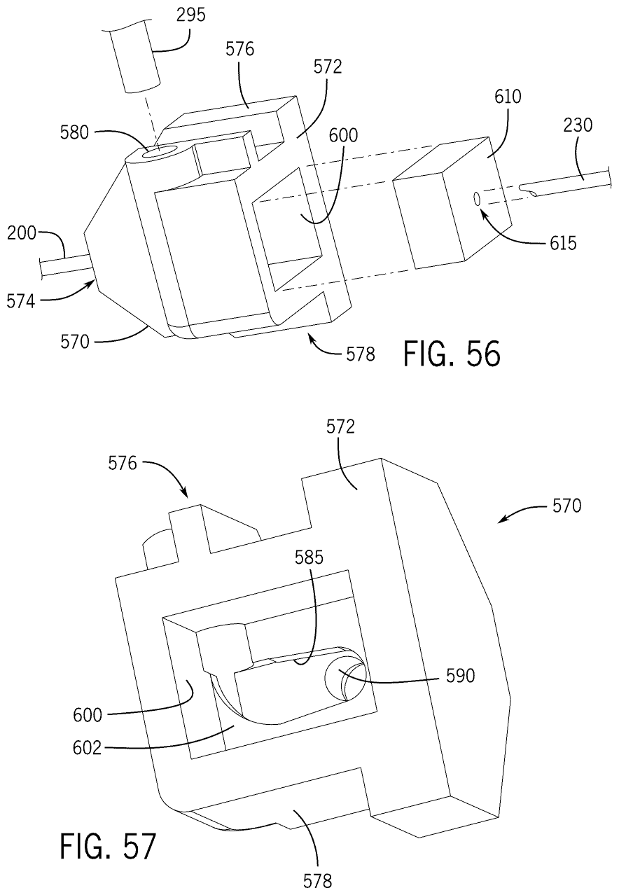

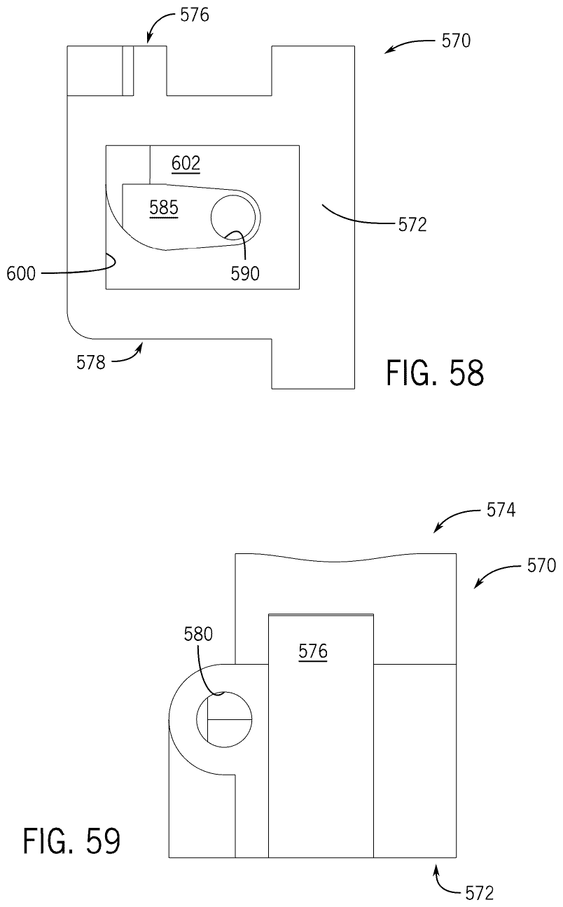

With reference also to FIGS. 55-59, retraction of the stylet 230 provides a clear flow path for liquid to be delivered from the reservoir to the cannula 200 and, in turn, to the patient. The second slide block 570 includes a first opening 580 configured to receive an end of the tubing 295 extending from the cartridge 35, or reservoir, to the second slide block. According to the illustrated embodiment, the first opening 580 is located on the upper surface 576 of the second slide block 570. The second slide block 570 includes a second opening 590 in which the first end of the cannula 200 is inserted. According to the illustrated embodiment, the second opening 590 is located on a front surface 574 of the second side block. The second slide block 570 also includes a third opening 600 on a rear surface 572 of the second slide block 570, where the rear surface 572 is on an opposite end of the second slide block from the front surface 574. According to the illustrated embodiment, the third opening 600 is square. An inner periphery of the third opening 600 has a pair of opposing side walls, an upper wall, and a lower wall. A seat 602 is defined on an interior surface of the third opening 600, where the seat 602 is a stop for an insert block 610 configured to be inserted into the third opening 600. The insert block 610 has a generally square outer periphery, complementary to the inner periphery of the third opening 600. The insert block 610 also includes a passage 615 extending through the insert block to receive the stylet 230. The illustrated embodiment is exemplary and not intended to be limiting. The opening 600 and insert block 610 may have various other geometric shapes, where the outer periphery of the insert block 610 is complementary to the inner periphery of the opening 600.

According to one aspect of the invention, the insert block 610 is made from an elastomeric material. The outer circumference of the insert block 610 may be greater than an inner circumference of the third opening 600 such that, as the insert block 610 is inserted into the third opening 600, the insert block 610 is compressed and/or elastically deformed to the front or back. The compression of the insert block 610 provides a sealing engagement of the insert block 610 to the third opening 600, preventing fluid from leaking out the third opening 600 during delivery of fluid to the patient. In addition to providing a sealing engagement of the insert block 610 to the third opening 600, the elastomeric material provides a sealing engagement around the stylet 230. The stylet 230 is inserted through the passage 615 in the insert block 610. Compression of the insert block 610 provides a sealing engagement of the insert block 610 around the stylet 230, preventing fluid from leaking out the passage 615 during delivery of fluid to the patient. Compression of the insert block 610 is not too great to prevent the stylet 230 from sliding within the insert block 610. Rather, the stylet 230 starts within the insert block 610 at the initial position shown in FIG. 51. As the stylet 230 is pushed forward through the lumen of the cannula 200 during insertion, the stylet 230 slides within the passage 615. Similarly, when the first slide block 630 moves back up the rail 562, the stylet slides within the passage 615 to a retracted position.

Turning next to FIG. 55, the stylet 230 is illustrated in a retracted position within the insert block 610 and within the second slide block 570. As indicated above, the insert block 610 is inserted into the third opening 600 of the second slide block 570 to seal the third opening 600. The insert block 610 is inserted until it engages the seat 602 on the interior of the third opening. The seat 602 is positioned at a depth within the second slide block 570 such that the insert block 610 does not block the first opening 580. Further, a passage 585 is molded on an interior surface of the second slide bock 570 to establish a fluid flow path between the first opening 580 and the second opening 590. During insertion, the stylet 230 is inserted in the lumen of the cannula 200. However, after retraction, the second end of the stylet 230 is positioned either within the passage 615 of the insert block 610 or just within the passage 585 between the first opening 580 and the second opening 590. Withdrawing the stylet 230 from the lumen of the cannula 200 increases the cross-sectional area for fluid to flow. As a result, fluids with greater viscosity are able to flow through the cannula 200 than may otherwise flow through the cannula 200 if the stylet 230 remains in the lumen. Further, the second end of the stylet 230 is removed into the passage 585 a sufficient distance so the stylet 230 does not reduce the flow between the first opening 580 and the second opening 590. Consequently, the diameter of the cannula 200 may be reduced, in comparison to a cannula with a stylet present, and permit the same flow rate, or the diameter of the cannula 200 may remain the same and permit delivery of fluids having a greater viscosity than may otherwise be delivered if the stylet were to remain in the cannula. Delivering medicament through a clear flow path reduces interference and allows for an increased flow rate or for a higher viscosity fluid to be delivered.

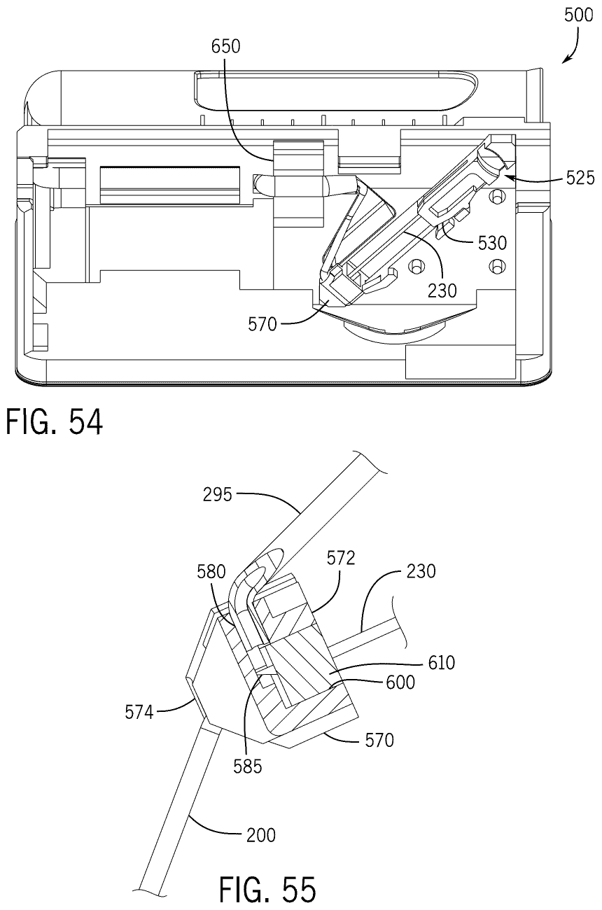

With reference also to FIG. 50 or 54, the needle injection mechanism 500 may include a sensor 650 configured to detect air in the tubing 295. According to one aspect of the invention, the sensor 650 is an ultrasonic sensor. The sensor 650 generates sound in the ultrasonic frequency range. The sound passes through the tubing 295 and is detected at a transducer. The ultrasonic sound travels differently through fluid than through air. As a result, detecting the time of flight between when the sound is emitted and when it is received may be utilized to detect air in the tubing 295. The control circuit 112 receives a feedback signal from the sensor 650. The control circuit 112 may monitor the feedback signal to determine if the air is temporary, such as a small air bubble, or of longer duration, such as when the reservoir, or cartridge 35 is empty. If too many small duration events occur, the control circuit 112 may detect an air-in-line fault condition and stop operation of the needle injection mechanism 500 to prevent excessive air from being injected into a patient. If the control circuit 112 detects an extended duration, it may determine that the cartridge 35 is empty and turn off the pump 330, completing an injection.

It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

We claim:

1. A wearable pump assembly, comprising:
a housing to be carried on a body of a patient;
a reservoir in fluid communication with the housing configured to hold a fluid for delivery from the pump assembly;
a pump mounted within the housing and configured to transfer the fluid from the reservoir to a cannula; and
a needle injection mechanism including:
a drive motor operative to rotate a drive shaft;
a drive train operative to transfer rotation of the drive shaft to a linear motion via a linkage;
a first slide block connected to the linkage to move along a guide member between a first position and a second position responsive to the linear motion, wherein the first slide block slidably moves a stylet through a lumen of the cannula while moving between the first position and the second position;
a second slide block pushed by the first slide block between a first position and a second position along the guide member; and
a retention tab made of a resilient material, wherein:
the retention tab is deflected from an initial position to a deflected position as the second slide block moves from the first position to the second position of the second slide block,
the retention tab returns to the initial position to hold the second slide block in the second position when the second slide block reaches the second position, and
the first slide block returns to the first position of the first slide block after pushing the second slide block to the second position of the second slide block, retracting the stylet from the lumen of the cannula.

2. The wearable pump assembly of claim 1, further comprising at least one tube extending between the reservoir and the second slide block and the second slide block includes:
a first opening in which the at least one tube is inserted;
a second opening in which the cannula is inserted; and
a passage extending between the first opening and the second opening to deliver the fluid from the at least one tube to the cannula.

3. The wearable pump assembly of claim 2, wherein:
the stylet is inserted through the passage and into the lumen of the cannula as the first slide block moves between the first position and the second position of the first slide block; and
a second end of the stylet is retracted from the passage when the first slide block returns to the first position of the first slide block and the second slide block is retained in the second position of the second slide block.

4. The wearable pump assembly of claim 2, wherein:
the second opening is on a front surface of the second slide block; and
the second slide block includes a third opening on a rear surface of the second slide block opposite the second opening; and the needle injection mechanism further comprises an insert block sized to fit the third opening, wherein:
the insert block includes a passage for the stylet,
the stylet slidably engages the passage in the insert block,
the insert block seals the third opening, and
the insert block provides a seal around the stylet as the stylet slides within the passage.

5. The wearable pump assembly of claim 2, wherein the pump is a peristaltic pump including a plurality of fingers operative to engage the at least one tube to draw the fluid from the reservoir to the second slide block.

6. The wearable pump assembly of claim 1, wherein the drive train of the needle injection mechanism further comprises:
a drive gear mounted to the drive shaft;
a plurality of additional gears sequentially driven from the drive gear; and
a flywheel coaxially mounted with one of the plurality of additional gears to rotate when the drive motor rotates the drive shaft.

7. The wearable pump assembly of claim 6 wherein the linkage is a drive arm having a first end pivotally mounted to the flywheel and a second end pivotally mounted to the first slide block.

8. The wearable pump assembly of claim 1, wherein the housing further comprises:
a first housing including a plurality of single-use devices; and
a second housing including a plurality of reusable devices.

9. The wearable pump assembly of claim 8, wherein the second housing is insertable into the first housing and a second end of the linkage engages the first slide block as the second housing is inserted into the first housing.

10. The wearable pump assembly of claim 1, wherein:
the reservoir is fillable, and
the housing includes a fill port in fluid communication with the reservoir.

11. The wearable pump assembly of claim 1, wherein:
the reservoir is a cartridge;
the housing includes a volume to receive the cartridge; and
the wearable pump assembly further comprises a cartridge adapter assembly to establish fluid communication between the cartridge and the second slide block.

12. A needle injection mechanism for a wearable pump assembly, comprising:
a drive motor operative to rotate a drive shaft;
a drive train operative to transfer rotation of the drive shaft to motion of a linkage, the linkage operative to convert rotational motion from the drive train to a linear motion;
a first slide block operative to move between a first position and a second position along a guide member responsive to the linear motion;
a stylet having a first end mounted to the fist slide block;
a second slide block operative to move between a first position and a second position along the guide member, wherein the first slide block engages the second slide block as the first slide block moves between the first position and the second position of the first slide block to drive the second slide block from the first position to the second position of the second slide block;
a cannula having a first end mounted to the second slide block and a lumen through which a second end of the stylet is slidably inserted; and
a retention tab made of a resilient material, wherein:
the retention tab is deflected from an initial position to a deflected position as the second slide block moves from the first position to the second position,
the retention tab returns to the initial to hold the second slide block in the second position when the second slide block reaches the second position, and
the first slide block returns to the first position after driving the second slide block to the second position, retracting the stylet from the cannula.

13. The needle injection mechanism of claim 12, wherein the second slide block includes:
a tube receiving a fluid from a reservoir of the wearable pump assembly;
a first opening in which an end of the tube is inserted;
a second opening in which the first end of the cannula is inserted; and
a passage extending between the first opening and the second opening to deliver the fluid from the tube to the cannula.

14. The needle injection mechanism of claim 13, wherein:
the stylet is inserted through the passage and into the lumen of the cannula as the first slide block moves between the first position and the second position; and
a second end of the stylet is retracted from the passage when the first slide block returns to the first position of the first slide block and the second slide block is retained in the second position of the second slide block.

15. The needle injection mechanism of claim 13, wherein:
the second opening is on a front surface of the second slide block; and
the second slide block includes a third opening on a rear surface of the second slide block opposite the second opening; and the needle injection mechanism further comprises an insert block sized to fit the third opening, wherein:
the insert block includes a passage for the stylet,
the stylet slidably engages the passage in the insert block,
the insert block seals the third opening, and
the insert block provides a seal around the stylet as the stylet slides within the passage.

16. The needle injection mechanism of claim 12, wherein the drive train further comprises:
a drive gear mounted to the drive shaft;
a plurality of additional gears sequentially driven from the drive gear; and
a flywheel coaxially mounted with one of the plurality of additional gears to rotate when the drive motor rotates the drive shaft.

17. The needle injection mechanism of claim 16 wherein the linkage is a drive arm having a first end pivotally mounted to the flywheel and a second end pivotally mounted to the first slide block.

18. The needle injection mechanism of claim 12, further comprising:
a first housing including a plurality of single-use devices; and
a second housing including a plurality of reusable devices, wherein the second housing is insertable into the first housing.

19. The needle injection mechanism of claim 18, wherein the second housing is insertable into the first housing and a second end of the linkage engages the first slide block as the second housing is inserted into the first housing.
